(19) United States
(12) Patent Application Publication
(10) Pub. No.: US 2025/0368695 A1
(43) Pub. Date: Dec. 4, 2025

(54) Atranorin biosynthesis gene derived from lichens and uses thereof

(71) Applicant: INDUSTRY-ACADEMIC COOPERATION FOUNDATION OF SUNCHON NATIONAL UNIVERSITY, Suncheon-si (KR)

(72) Inventors: Jae Seoun HUR, Suncheon-si (KR); Won Yong KIM, Suncheon-si (KR); Hyung Ho HA, Suncheon-si (KR); Hangun KIM, Gwangju (KR)

(21) Appl. No.: 18/567,890

(22) PCT Filed: Jun. 7, 2022
(86) PCT No.: PCT/KR2022/008015

§ 371 Date: Dec. 7, 2023

(30) Foreign Application Priority Data
Jun. 7, 2021 (KR) 10-2021-0073669
May 16, 2022 (KR) 10-2022-0059678

(51) Int. Cl. C07K 14/41; C12N 1/14; C12N 15/80; C12P 7/62; C12R 1/645

(52) U.S. Cl. CPC C07K 14/41; C12N 1/145; C12N 15/80; C12P 7/62; C12R 2001/645

(57) ABSTRACT

The present invention relates to an atranorin biosynthesis gene derived from a lichen Stereocaulon alpinum, an Ascochyta rabiei ATR-11 strain (Accession Number: KACC 83048BP) for producing atranorin into which the gene is introduced, and a method of producing atranorin using the above strain. A lichen-derived metabolite, particularly atranorin, may be produced from the Ascochyta rabiei ATR-11 strain into which the atranorin biosynthesis gene according to the present invention is introduced. Atranorin produced from the above strain can be utilized and industrialized in a variety of ways, such as pharmaceutical compositions, food compositions, health functional foods, and feed compositions.

### REFERENCE TO A SEQUENCE LISTING

The contents of the electronic sequence listing (JPK20230113US_sequence_listing_revised.txt; Size: 45,848 bytes; and Date of Creation: Mar. 6, 2025) is herein incorporated by reference in its entirety. The contents of the electronic sequence listing in no way introduces new matter into the specification.

### TECHNICAL FIELD OF THE INVENTION

The present invention relates to an atranorin biosynthesis gene derived from a lichen and a use thereof. More specifically, it relates to an atranorin biosynthesis gene derived from Stereocaulon alpinum, an Ascochyta rabiei ATR-11 strain (Accession Number: KACC 83048BP) for producing atranorin into which the gene is introduced, and a method of producing atranorin using the above strain.

### BACKGROUND ART OF THE INVENTION

Lichens play significant roles in ecosystem function and comprise about 20% of all known fungi. Polyketide-derived natural products accumulate in the cortical and medullary layers of lichen thalli, some of which play key roles in protection from biotic and abiotic stresses (herbivore attacks and UV irradiation). To date, however, no single lichen product has been linked to respective biosynthetic genes with genetic evidence.

Lichen-forming fungi (LFF) live in symbiosis with photosynthetic partners, green algae, or cyanobacteria, sometimes with both, and LFF are currently estimated to comprise about 20% of all known Fungi. Lichen symbiosis is one of the most successful mutualisms that enable these organisms to adapt to extremely harsh habitats. A conglutinated, often pigmented, cortical layer made up of dense fungal hyphae provides photosynthetic partners with mechanical stabilization, and algal cells enveloped by hyphae from the medulla provide nutrients to LFF by means of photosynthesis. Secondary metabolites (SMs) of polyketide origin, namely anthraquinones, depsides, depsidones, and dibenzofurans (such as usnic acid), accumulate in cortical or medullary layers of lichen thalli (Calcott M J et.al., Chem Soc Rev., 47:1730-1760, 2018). The ecological roles of these lichen SMs are largely unknown, but some studies provide evidence that cortical substances have contributed to habitat expansion and defense from herbivore attacks.

Depsides and depsidone series compounds are widespread in lichens as cortical and medullary substances, many of which are exclusively found in lichens. Lichen depsides are formed by dimerization of either orsellinic acid or 3-methylorsellinic acid (3MOA), and subsequent oxidation of depsides affords tricyclic scaffolds for depsidones. These compounds further undergo different combinations of modification within orsellinic acid and 3MOA moieties, such as alkylation, chlorination, hydroxylation, and O-methylation, yielding hundreds of structurally diverse compounds. For this, depsides/depsidones biosynthetic pathways in lichens could be a model system for studying substrate specificity of biosynthetic enzymes resulting in chemodiversity. Thus far, however, ascribing specific biosynthetic genes to cortical or medullary substance has been slow in lichens, due to a paucity of genetic information on LFF and lack of molecular tools for manipulating LFF recalcitrant to genetic transformation.

Non-reducing iterative type I polyketide synthases (NR-PKSs) are multi-domain enzymes and has been grouped into seven or eight major groups by protein sequence similarity and PKS domain architecture. Orsellinic acid and 3MOA are basic scaffolds for many different SMs in bacteria, fungi and plants, and biosynthesized by NR-PKSs in a filamentous fungus, Aspergillus nidulans, yet only predictions have been made for two NR-PKSs to be responsible for the biosynthesis of the basic units in lichens. Atranorin (a 3MOA-derived depside) and usnic acid (a dibenzofuran) are the most common cortical substances of macrolichens, and have attracted great attention because of their taxonomic, ecological, and pharmaceutical importance. Several studies have ascribed an NR-PKS to usnic acid with high likelihood. However, the effort was unsuccessful for heterologous expression of the putative usnic acid PKS gene in A. oryzae, and thus its precise function remains to be determined.

In recent years, SM research has benefited extensively from genome mining approaches in bacteria, fungi, and plants Antarctic lichens Cladoniaceae is one of the largest families of LFF and closely related to Stereocaulaceae. Historically, chemotaxonomy has been used as a polyphasic approach to resolve and delimit species boundaries in Cladoniaceae, and the genus Cladonia has been a model for studying PKS genes for the biosynthesis of cortical and medullary substances (Armaleo D et.al., Mycologia, 103:741-754, 2011).

The present inventors classified lichen polyketide synthases (PKS) and reconstructed the biosynthetic pathway of atranorin, which is the main cortical substance of lichen, in a heterologous host. Accordingly, the present inventors completed the present invention by securing a gene responsible for the biosynthesis of atranorin, a lichen cortical material, and constructing a new heterologous expression system into which the gene is introduced and developing a new strain for producing atranorin.

### PRIOR ART DOCUMENTS

### Non-Patent Documents

(1) Calcott M J et.al., Chem Soc Rev., 47:1730-1760, 2018
(2) Armaleo D et.al., Mycologia, 103:741-754, 2011

### DISCLOSURE

### Technical Problem

An object of the present invention is to provide an atranorin biosynthesis gene consisting of one or more selected from the group consisting of SEQ ID NO: 1, SEQ ID NO: 3, and SEQ ID NO: 5.

Another object of the present invention is to provide a recombinant expression vector for producing metabolite derived from a lichen, including the atranorin biosynthesis gene.

Still another object of the present invention is to provide a recombinant microorganism transformed with the recombinant expression vector.

Yet another object of the present invention is to provide a method of producing a metabolite derived from lichens, includes a step of culturing the recombinant microorganism.

Yet another object of the present invention is to provide Ascochyta rabiei ATR-11 strain (Accession Number: KACC 83048BP).

Yet another object of the present invention is to provide a method of producing atranorin, including a step of culturing the strain.

### Technical Solution

In order to achieve the above object, the present invention provides an atranorin biosynthesis gene consisting of one or more species selected from the group consisting of SEQ ID NO: 1, SEQ ID NO: 3, and SEQ ID NO: 5.

Additionally, the present invention provides a recombinant expression vector for producing metabolite derived from a lichen, including the atranorin biosynthesis gene.

Additionally, the present invention provides a recombinant microorganism transformed with the recombinant expression vector.

Additionally, the present invention provides a method of producing a metabolite derived from lichens, includes a step of culturing the recombinant microorganism.

Additionally, the present invention provides Ascochyta rabiei ATR-11 strain (Accession Number: KACC 83048BP).

Additionally, the present invention provides a method of producing atranorin, including a step of culturing the strain.

### Advantages Effects

A heterologous expression system into which a lichen-derived atranorin biosynthesis gene according to the present invention is introduced is constructed, and lichen-derived metabolites, especially atranorin, can be biosynthesized from the Ascochyta rabiei ATR-11 strain by applying the same. Atranorin produced from the above strain can be utilized and industrialized in a variety of ways, such as pharmaceutical compositions, food compositions, health functional foods, and feed compositions.

### BRIEF DESCRIPTION OF THE DRAWINGS

FIG. 1 shows the genome-encoded metabolic potentials of lichen. A coalescent-based species tree is reconstructed for 29 lichen-forming fungi and Cyanodermella asteris (a plant endophyte). The tree is rooted to Endocarpon pusillum. All local posterior probabilities for nodal supports are higher than 0.98, except those marked by arrows. The five orders of the Lecanoromycetes are highlighted with different colors, and lichen genomes sequenced by Korean Lichen Research Institute are marked in bold. BUSCO describes the completeness of the genome's assemblies. The number of biosynthetic gene clusters (BGCs) are predicted by antiSMASH, and bubble plots indicate relative abundance of core biosynthetic enzyme categories: NR-PKS, non-reducing type I PKS; R-PKS, reducing type I PKS (including PKS-NRPS hybrid enzymes); PKSIII, type III PKS; NRPS, nonribosomal peptide synthetase (including NRPS-like enzymes); and Terpene, biosynthetic enzymes related to terpenoids production.

FIG. 2 shows Polyketide BGC diversity in Cladonia species. (A) Forty-five gene cluster families (GCFs) were identified by gene cluster network analysis in the six Cladonia spp. And Stereocaulon alpinum. GCFs are labelled with their associated PKS families. Numbers in red indicate PKS families connected to characterized BGCs deposited in the MIBiG database. Nodes indicate BGCs and are color-coded by species (see the inset). Nodes enclosed by a red dashed circle in the PKS8, PKS14 and PKS16 subnetworks are lichen BGCs in the MIBiG database. Edge width was drawn proportional to squared similarity between a pair of BGCs. Singleton BGCs that connected to known BGCs are labelled as follows: M, monascorubrin BGC; H, hypothemycin BGC; S, sorbicillin BGC; T, terreic acid BGC; B, betaenones BGC; D, depudecin BGC; C, curvupallides BGC. (B) Phyletic distribution of PKS families evidenced by gene cluster network analysis. Numbers in violet and ochre indicate NR-PKS and R-PKS families, respectively. Black squares indicate the presence of PKS families, and grey squares indicate pseudogenes or partial genes due to incomplete gene annotation. Signature secondary metabolites linked to Cladonia PKS families by genetic dereplication are marked in bold.

FIG. 3 shows an organization of putative atranorin BGCs. Fifteen BGCs including an O-methyltransferase that shows significant hits to the one in the PKS23 BGC in C. rangiferina (Crg, labeled in red) are identified from 1,527 BGCs detected in the 30 genomes, using the CORASON analysis pipeline. Lichen species that harbor the BGCs are labelled with their abbreviated names, as in FIG. 1. Numbers in blue indicate percent protein sequence identity of homologous OMTs to the one in Crg. Among the 15 BGCs, seven BGCs harbor four synthetic genes (shaded box): atr1 (PKS23), atr2, atr3 and atr4. Genes related to secondary metabolism are color-coded, based on their predicted function. NR-PKS, non-reducing type I PKS; R-PKS, reducing type I PRS; NRPS, nonribosomal peptide synthetase; TF, transcription factor; RTA, resistance to 7-aminocholesterol.

FIG. 4 shows the results of functional validation of atranorin BGC. (A) A proposed biosynthetic pathway for atranorin. (B) HPLC profiles of culture extracts of a “clean host” expressing different sets of biosynthetic genes of the atranorin BGC: with no introduced gene (i), with atr1 only (ii), with atr1 and atr3 (iii), with atr1 and atr2 (iv), and with atr1, atr2, and atr3 (v). The acetone extract of an authentic voucher specimen for the genome sequenced Stereocaulon alpinum contains atranorin and lobaric acid (t_{R}=33.1 min) (vi). (C) Extracted ion chromatograms (EIC) with the indicated m/z values for the compounds 1-6.

FIG. 5 shows the ninth clade of fungal non-reducing PKS genes. A maximum likelihood tree of 103 NR-PKSs in the six Cladonia spp. and Stereocaulon alpinum and 82 NR-PKSs linked to known compounds in non-lichenized fungi is reconstructed using concatenated sequences of ketosynthase and product template domains. Outgroup was set to 6-methylsalicylic acid synthases (6MSAS) found in Cladonia spp. and the 6MSAS for the patulin biosynthesis in Penicillium expansum. Branches are color-coded, based on bootstrap (BS) values. The scale represents 0.5 amino acid sequence substitutions per site. Blue and red strips with associated numbers at the outermost region indicate PKS families identified by the gene cluster network analysis (see FIG. 2). Phylogenetic clades representing nine NR-PKS groups (Groups I-IX) are shaded with different colors. The NR-PKS groups are supported by BS values greater than 75, except for the Group II. Note that the newly identified NR-PKS group (Group IX) includes the PKS1, PKS2, and PKS23 families, candidate PKSs for cortical and medullary substances of lichens. Red arrows indicate polyphyletic losses of C-methyltransferase domain of NR-PKSs.

FIG. 6 shows chemical structures of cortical and medullary substances of lichens. Lichen substances reported in the 30 genome-sequenced species are grouped by nine chemical groups, based on the chemical structures (Also, see Table 2). Here, 3MOA is an abbreviation for 3-methylorsellinic acid.

FIG. 7 shows the results of verification of heterologous expression of PKS23. (A) A schematic diagram of the PKS23 (atr1) gene structure in Stereocaulon alpinum. Black boxes are exons, and grey lines are introns. The numbers indicate the size of exons and introns in base pairs (bp). P1 and P2 are primer pairs used in RT-PCR analyses. These primer pairs are designed to include one or more intron regions so that mRNA expression can be distinguished from genomic DNA (gDNA) amplification. (B) RT-PCR analyses of two putative transformants (T16 and T25) carrying the plasmid sol1::atr1/pDS35. No band corresponding to gDNA amplicon for Actin1 reference gene (633 bp; cf. 482 bp for mRNA) is observed, indicating that there was no genomic DNA contamination (lower left panel). The transformant T25 shows stronger expression of the introduced atr1 than the transformant T16 (upper left panel). Splicing of the four introns in the atr1 is confirmed by comparison of band sizes between the plasmid sol1::atr1/pDS35 (C) and RNA sample (T25), using P1 and P2 primer pairs. The expected band sizes for mRNA expression and plasmid DNA amplification are indicated by numbers to left and right sides of the gel image in bp. Here, M is a 100 bp DNA ladder.

### DETAILED DESCRIPTION

Depsides and depsidones series compounds of polyketide origin accumulate in the cortical or medullary layers of lichen thalli. Despite the taxonomic and ecological significance of lichen chemistry and its pharmaceutical potentials, there has been no single genetic evidence linking biosynthetic genes to lichen substances. Thus, the present inventors systematically analyzed lichen polyketide synthases (PKSs) for categorization and identification of biosynthetic gene cluster (BGC) involved in depside/depsidone production. Our in-depth analysis of the inter-species PKS diversity in the genus Cladonia and a related Antarctic lichen Stereocaulon alpinum identified 45 BGC families, linking lichen PKSs to 15 previously characterized PKSs in non-lichenized fungi. Among these, the present inventors identified highly syntenic BGCs found exclusively in lichens producing atranorin (a depside). Heterologous expression of the putative atranorin PKS (coined atr1) yielded 4-O-demethylbarbatic acid found in many lichens as a precursor compound, indicating an intermolecular cross-linking activity of the Atr1 for depside formation. Subsequent introductions of tailoring enzymes into the heterologous host yielded atranorin, one of the most common cortical substances of microlichens. Phylogenetic analysis of fungal PKS revealed that the Atr1 is placed in a novel PKS clade including two conserved lichens specific PKS families likely involved in biosynthesis of depsides and depsidones. Here, the present inventors provide a comprehensive catalog of PKS families of the genus Cladonia and functionally characterized a biosynthetic gene cluster from lichens, establishing a cornerstone for studying genetics and chemical evolution of diverse lichen substances.

### Novel Atranorin Biosynthesis Gene

As one aspect, the present invention provides an atranorin biosynthesis gene consisting of one or more selected from the group consisting of SEQ ID NO: 1, SEQ ID NO: 3, and SEQ ID NO: 5.

The term “atranorin” used in the present specification has a chemical formula name of 3-hydroxy-4-methoxycarbonyl-2,5-dimethylphenyl-3-formyl-2,4-dihydroxy-6-methyl benzoate, and has a structural formula of [Formula 1] shown below. Such atranorin may be derived from thalli as a secondary metabolite of thalli. The atranorin has been reported to have an effect of inhibiting lung cancer metastasis.

### [Formula 1]

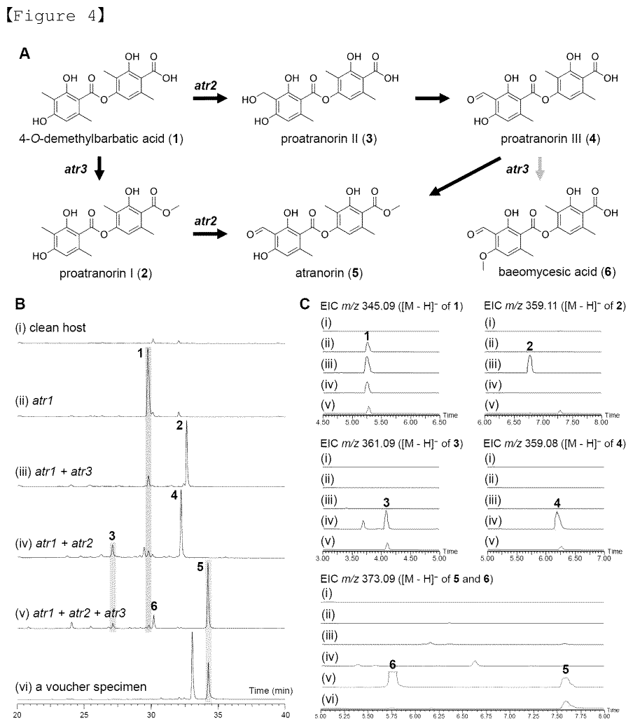

See atranorine (compound 5) in FIG. 4A.

Atranorin of the present invention may be used in a meaning of including analogues and derivatives of atranorin.

The term “thallus” used in the present specification refers to a trophosome of lichens for seredium, isidium or clonal propagation, consisting of cells of lichen symbiotic algae or cyanobacteria and hyphae of a lichen-forming fungus.

The term “lichen” used in the present specification refers to a symbiont living as a complex of fungi (mycobionts) and algae (photobionts), and it has extremely diverse shapes, sizes, and colors, inhabiting mainly on rocks, barks of tree, and soil. The lichens produce primary and secondary metabolites such as didymic acid, strepsilin, sodium usnate, lecanoric acid, and psoromic acid which have excellent bioactivity for antimicrobials (fungi, bacteria, viruses), herbicides, anti-cancer agents, immunomopotentiation, and amelioration of metal illnesses. Therefore, from old times, lichens have been traditionally used as a raw material for foods and medicines in both East and West of the world.

In the present invention, the atranorin biosynthesis gene is a secondary metabolite derived from a lichen, for example, a gene capable of inducing the expression of atranorin, and may consists of one or more selected from the group consisting of SEQ ID NO: 1, SEQ ID NO: 3, and SEQ ID NO: 5. The lichen may be Stereocaulon alpinum but is not limited thereto.

The base sequence represented by SEQ ID NO: 1 may be named as atr1 gene (NCBI Genbank No. MZ277879.1); the base sequence represented by SEQ ID NO: 3 as atr2 gene (NCBI Genbank No. MZ277878.1); and the base sequence represented by SEQ ID NO: 5 as atr3 gene (NCBI Genbank No. MZ277877.1).

In addition, the base sequence represented by SEQ ID NO: 1 may be encoded as an atr1 protein (NCBI Genbank No. QXF68953.1) consisting of an amino acid sequence of SEQ ID NO: 2. The base sequence represented by SEQ ID NO: 3 may be encoded as an atr2 protein (NCBI Genbank No. QXF68952.1) consisting of an amino acid sequence of SEQ ID NO: 4. The base sequence represented by SEQ ID NO: 5 may be encoded as an atr3 protein (NCBI Genbank No. QXF68951.1) consisting of an amino acid sequence of SEQ ID NO: 6.

### Recombinant Expression Vector for Producing Lichen-Derived Metabolites.

As another aspect, the present invention provides a recombinant expression vector for producing a metabolite derived from a lichen, including an atranorin biosynthesis gene consisting of one or more selected from the group consisting of SEQ ID NO: 1, SEQ ID NO: 3, and SEQ ID NO: 5.

The “atranorin biosynthesis gene” is as described above.

The term “vector” used in the present specification refers to a DNA preparation containing a specific gene operably linked to a suitable regulatory sequence to enable expression of a target protein in a suitable host, wherein the regulatory sequence includes a promoter capable of initiating transcription, an arbitrary operator sequences for regulating the transcription, a sequence encoding a suitable mRNA ribosome binding site, and a sequence regulating the termination of transcription and translation. After being transformed into a suitable host cell, a vector may be replicated or may function independently of the host genome, and it may be integrated into the genome itself. The vector of the present invention may be prepared using a genetic recombination technology well known in the technical field, and enzymes generally known in the technical field are used for site-specific DNA cleavage and ligation.

The term “expression vector” used in the present specification refers to a vector into which a lichen-derived atranorin biosynthesis gene is introduced, wherein it means a recombinant expression vector capable of expressing a lichen-derived metabolite, prepared by introducing a lichen-derived atranorin biosynthesis gene to a heterologous strain, in order to artificially express lichen-derived metabolites that may not be expressed in heterologous microorganisms or host cells on their own.

Specifically, the expression vector means a vector capable of expressing a lichen-derived metabolite such as atranorin, which is a secondary metabolite derived from lichens, as well as 4-O-demethylbarbatic acid, proatranorin I, proatranorin II, proatranorin III, and baeomycesic acid, in heterologous microorganisms or host cells by the introduced atranorin biosynthesis gene. In the present invention, an expression vector may be used interchangeably with ‘vector’ or ‘recombinant expression vector.

The expression vector may preferably include one or more selectable markers. The marker is a nucleic acid sequence that has the characteristics that it may be selected by a conventional chemical method and includes all genes that may distinguish transformed cells from non-transformed cells. Examples include antibiotic resistance genes such as ampicillin, kanamycin, G418, bleomycin, hygromycin, chloramphenicol, and apramycin, but are not limited thereto, and it may be appropriately selected by one of ordinary skill in the art.

In one embodiment of the present invention, a vector into which an atranorin biosynthesis gene derived from Stereocaulon alpinum is introduced may be transformed into a heterologous chickpea blight fungus Ascochyta rabiei to heterologously express lichen-derived metabolites.

Recombinant Microorganism Loaded with a Recombinant Expression Vector Containing an Atranorin Biosynthetic Gene

In another aspect, the present invention provides a recombinant microorganism transformed with the above recombinant expression vector.

The recombinant expression vector is as described above.

The term “transformation” used in the present specification refers to a change in the genetic properties of an organism by an externally given DNA. In particular, in the present invention, it refers to introducing a vector including a specific gene, that is, a lichen-derived atranorin biosynthesis gene, to a specific heterologous microorganism so that the gene may be expressed in the specific heterologous microorganism.

The recombinant expression vector is transformed into a specific heterologous microorganism to produce a recombinant microbial strain. At this time, the transformation method may be used without any particular limitation as long as it is a known technology. For example, a calcium chloride method or an electroporation method (Neumann, et al., EMBO J., 1:841, 1982) may be used for transformation.

The specific heterogeneous microorganism may be used without limitation as long as it is a microorganism or host cell that widely known in the art. Microorganisms or host cells with a high efficiency of introducing and expressing a lichen-derived atranorin biosynthesis gene of the present invention may be used, and for example, they may include fungi, bacteria, and yeast.

Specifically, the recombinant microorganism may be a fungus, and more specifically, it may be Ascochyta rabiei.

More specifically, the recombinant microorganism may be Ascochyta rabiei ATR-11 strain (Accession Number: KACC83048BP).

### Method of Producing a Lichen-Derived Metabolite

In another aspect, the present invention provides a method of producing a lichen-derived metabolite, including a step of culturing the recombinant microorganism.

As described above, the recombinant microorganism may be a recombinant microorganism into which a recombinant expression vector for producing a lichen-derived metabolite, including an atranorin biosynthesis gene consisting of one or more selected from the group consisting of SEQ ID NO: 1, SEQ ID NO: 3, and SEQ ID NO: 5, is introduced.

The recombinant microorganism may be one producing one or more lichen-derived metabolites selected from the group consisting of 4-O-demethylbarbatic acid, proatranorin I, proatranorin II, proatranorin III, atranorin, and baeomycesic acid, but is not limited thereto.

Culture conditions of the recombinant microorganism for producing a lichen-derived metabolite may vary depending on the type of transformed recombinant microorganism or host cell. Depending on the type of the recombinant microorganism or host cell, culture may be performed according to conditions known to one of ordinary skill in the art, that is under conventional temperature conditions of, for example, 25 to 40° C., and under conventional pH conditions, for example, pH 6 to 8. Under these conditions, the above lichen-derived metabolites may be mass-produced from a recombinant microorganism or host cell with a high yield.

As described above, as the recombinant microorganism or hos cell, microorganisms or host cells known in the art with a high efficiency of introducing and expressing a lichen-derived atranorin biosynthesis gene may be used without a limitation. Specifically, the recombinant microorganism may be a fungus, and more specifically, it may be Ascochyta rabiei. More specifically, the recombinant microorganism may be Ascochyta rabiei ATR-11 strain (Accession Number: KACC 83048BP).

Additionally, as a medium used for culturing (or fermenting) the recombinant microorganism or host cell, one that appropriately satisfies the requirements of a specific microbial strain or host cell may be appropriately selected. The medium may include various carbon sources, nitrogen sources, phosphorus sources, and trace element components. Examples of carbon sources in the medium may be sugars and carbohydrates such as glucose, saccharose, lactose, fructose, maltose, starch, and cellulose, oils, and fats such as soybean oil, sunflower oil, castor oil, and coconut oil, fatty acids such as palmitic acid, stearic acid, and linoleic acid, alcohols such as glycerol and ethanol, and organic acids such as acetic acid, but are not limited thereto. These substances may be used individually or in mixtures. The carbon source may be glycerol, glucose, or a combination thereof. Examples of nitrogen sources in the medium may be peptone, yeast extract, broth, malt extract, corn steep liquor, soybean meal, and urea or inorganic compounds, for example, ammonium sulfate, ammonium chloride, ammonium phosphate, ammonium carbonate, and ammonium nitrate, but are not limited thereto. Nitrogen sources may also be used individually or in mixtures. Examples of phosphorus sources may be potassium dihydrogen phosphate or dipotassium hydrogen phosphate or corresponding sodium-containing salts but are not limited thereto. In addition, a culture medium may include metal salts necessary for growth, such as magnesium sulfate or iron sulfate, or may include essential growth substances such as amino acids and vitamins but is not limited thereto. The above raw materials may be added to a culture batchwise or continuously in an appropriate manner during a culture process.

Additionally, according to the necessary, the pH of the culture may be adjusted by using basic compounds such as sodium hydroxide, potassium hydroxide, and ammonia, or acid compounds such as phosphoric acid or sulfuric acid in an appropriate manner. In addition, foam generation may be suppressed by using an antifoaming agent such as fatty acid polyglycol ester. The temperature of the culture may usually be maintained at 20° C. to 45° C., or 25° C. to 40° C. The culture may be performed continuously until the maximum production of a desired metabolite, for example, one or more lichen-derived secondary metabolites selected from the group consisting of 4-O-dimethylbarbatic acid, proatranorin I, proatranorin II, proatranorin III, atranorin, and baeomycesic acid, is obtained.

The method of producing a lichen-derived metabolite provided in the present specification may further include a step of recovering or isolating a produced lichen-derived secondary metabolite after the step of culturing described above. A method of recovering or isolating a lichen-derived secondary metabolite produced from a recombinant strain or a culture thereof may be selected from methods that are widely known in the art. Examples of methods of recovering 4-O-dimethylbarbatic acid, proatranorin I, proatranorin II, proatranorin III, atranorin or baeomycesic acid include centrifugation, sonication, filtration, ion exchange chromatography, high-performance liquid chromatography (HPLC), gas chromatography (GC), and the like, but are not limited to these examples.

The lichen-derived metabolites provided in the present specification may be produced from a recombinant microorganism transformed by selectively introducing one or more of the above-mentioned atranorin biosynthesis genes (see Scheme 1 below).

### [Scheme 1]

### See FIG. 4A.

In one embodiment of the present invention, 4-O-dimethylbarbatic acid may be produced by culturing under predetermined conditions a recombinant microorganism into which the atr1 gene consisting of the base sequence of SEQ ID NO: 1 is introduced. At this time, the recombinant microorganism is not limited thereto, but may be phytopathogenic fungus Ascochyta rabiei (As. rabiei).

In one embodiment of the present invention, proatranorin I may be produced by culturing under predetermined conditions a recombinant microorganism into which the atr1 gene consisting of the base sequence of SEQ ID NO: 1 and the atr3 gene consisting of the base sequence of SEQ ID NO: 5 are introduced. At this time, the recombinant microorganism is not limited thereto, but may be phytopathogenic fungus Ascochyta rabiei (As. rabiei).

In one embodiment of the present invention, proatranorin II and proatranorin III may be produced by culturing under predetermined conditions a recombinant microorganism into which the atr1 gene consisting of the base sequence of SEQ ID NO: 1 and the atr2 gene consisting of the base sequence of SEQ ID NO: 3 are introduced. At this time, the recombinant microorganism is not limited thereto, but may be phytopathogenic fungus Ascochita rabiei (As. rabiei).

In one embodiment of the present invention, atranorin may be produced by culturing under predetermined conditions a recombinant microorganism into which the atr1 gene consisting of the base sequence of SEQ ID NO: 1, the atr2 gene consisting of the base sequence of SEQ ID NO: 3, and the atr3 gene consisting of the base sequence of SEQ ID NO: 5 are introduced. At this time, the recombinant microorganism is not limited thereto, but may be phytopathogenic fungus Ascochita rabiei (As. rabiei).

Ascochyta rabiei ATR-11 Strain for Producing Atranorin

As another aspect, the present invention provides Ascochyta rabiei ATR-11 strain (Accession Number: KACC83048BP).

The term “Ascochyta rabiei” used in the present specification refers a phytopathogenic fungus that causes chickpea blight, and it is a heterologous host microorganism used to introduce the lichen-derived atranorin biosynthesis gene described above.

The Ascochyta rabiei ATR-11 strain (Accession Number: KACC83048BP) is a strain for producing atranorin, deposited to the Korean Agricultural Culture Collection (KACC) of the National Institute of Agricultural Sciences on May 28, 2021, with the Accession Number KACC 83048BP.

Specifically, the Ascochyta rabiei ATR-11 strain is the above-described strain into which an atranorin biosynthesis gene consisting of one or more selected from the group consisting of SEQ ID NO: 1, SEQ ID NO: 3, and SEQ ID NO: 5 is introduced, and it is capable of producing atranorin. Atranorin produced from the above strain can be utilized and industrialized in a variety of ways, such as pharmaceutical compositions, food compositions, health functional foods, and feed compositions.

Method of Producing Atranorin Using Ascochyta rabiei ATR-11 Strain

As another aspect, the present invention provides a method for producing atranorin, including a step of culturing Ascochyta rabiei ATR-11 strain (Accession Number: KACC83048BP).

The Ascochyta rabiei ATR-11 strain (Accession Number: KACC 83048BP) is as described above.

The culture conditions of the Ascochyta rabiei ATR-11 strain (Accession Number: KACC 83048BP) may be a temperature of 15 to 25° C. and a culture duration of 7 to 21 days, preferably, a temperature of 18 to 22° C. and a culture duration of 14 to 18 days, but are not particularly limited thereto.

In addition, culturing of the Ascochyta rabiei ATR-11 strain (Accession Number: KACC 83048BP) may be performed by using a natural medium or synthetic medium including carbon sources, nitrogen sources, inorganic salts, and the like that may be efficiently utilized by the strain. Carbon sources that may be used include carbohydrates such as glucose, fructose, sucrose, soybean powder, peanut powder, wheat flour, corn powder, dextrin, corn meal, and fructose syrup; starch and hydrolysate of starch; organic acids such as acetic acid and propionic acid; alcohols such as ethanol, propanol, and glycerol; and oils such as soybean oil, olive oil, canola oil, peanut oil, and fish oil. Nitrogen sources include ammonia; ammonium salts of inorganic or organic acids such as ammonium chloride, ammonium sulfate, ammonium acetate, and ammonium phosphate; peptone, meat extract, yeast extract, corn steeping liquid, casein hydrolysate, soybean extract, soybean hydrolysate; and various fermented cells and decomposition products thereof. Amino acids include monosodium glutamate, methionine, lysine, leucine, cysteine, and valine. Inorganic salts include potassium dihydrogen phosphate, dipotassium hydrogen phosphate, magnesium phosphate, magnesium sulfate, sodium chloride, manganese sulfate, copper sulfate, calcium carbonate, and the like.

The pH of the medium is preferably maintained in a range of 3.0 to 9.0 during culture. The pH of the medium may be adjusted with inorganic or organic acids, alkaline solutions, urea, calcium carbonate, ammonia, and the like.

After culturing the Ascochyta rabiei ATR-11 strain (Accession Number: KACC 83048BP), various known methods may be used to isolate, purify, and recover atranorin therefrom. For example, after removing the bacterial cells by centrifugation, isolation and purification may be performed by appropriate known methods such as salting out, ethanol precipitation, ultrafiltration membrane, gel filtration chromatography, ion exchange column chromatography, or a combination thereof.

Production of atranorin using the Ascochyta rabiei ATR-11 strain (Accession Number: KACC 83048BP) is suitable for not only lab-scale production but also large-scale production, and thus is to be commercially applied.

The atranorin may be atranorin derived from a lichen Stereocaulon alpinum but is not limited thereto.

### EXAMPLES

Hereinafter, the constitution and effects of the present invention will be described in more detail through examples. These examples are only for illustrating the present invention, and the scope of the present invention is not limited by these examples.

### Example 1: Phylogenomic Analysis

To infer the phylogeny of the genome-sequenced 29 LFF (Lichen-forming fungi) and an endophytic fungus Cyanodermella asteris, the present inventors pursued a coalescent-based phylogenomic approach. The culture, genome sequencing and annotation procedures for LFF were described in Example 8. Single-copy ortholog clusters (SCOs) of protein sequences deduced from the 30 annotated genomes were identified using OrthoMCL (v2.0.9) (64) with an inflation factor of 2.5. For each of the 393 SCOs, protein sequences were aligned using MAFFT (v7.310) (65) with the ‘auto’ setting, and the resulting 393 multiple sequence alignments were trimmed for poorly aligned regions using Gblocks (v0.91b) (66) with the parameter: ‘−b4=5’. The RA×ML program (v8.2) (67) was used to calculate 100 maximum likelihood trees for each multiple sequence alignment. To generate coalescent-based trees, the present inventors used ASTRAL-III (v5.7.4) (68) with two multi-locus bootstrapping options (site only resampling and gene/site resampling) and with no bootstrapping option. All trees showed the identical topology.

### Example 2: Biosynthetic Gene Cluster Family Analysis

For BGC identification in the 30 genomes, the genome assembly and annotation files were processed by the antiSMASH program (v5.0+), with a parameter setting: ‘--minimal’. For genetic dereplication of BGCs found in the six Cladonia spp. and S. alpinum, the present inventors used the BiG-SCAPE program, in reference to the MIBiG database (v1.4). To analyze BGCs containing at least one iterative type I PKS, the present inventors modified a configuration file ‘domain_whitelist.txt’ to include only Pfam domains related to iterative type I PKS (PF00109 and PF02801) and ran the BiG-SCAPE program with optional arguments: ‘--mix’ and ‘--hybrids-off’. Based on the Jaccard index of domain types, domain sequence similarity, and domain adjacency index, the BiG-SCAPE program calculates a similarity matrix between pairwise combinations of clusters where smaller values indicate greater BGC similarity. The present inventors evaluated BGC networks using an edge-length cutoff from 0.3-0.8 with a step of 0.1 and considered the network using a cutoff value of 0.5 as a representation of GCFs in the six Cladonia spp. and S. alpinum. Individual networks for PKS families were visualized, using a Python package NetworkX (v2.5), in that edge-width was weighted by squared similarity between a pair of BGCs calculated by BiG-SCAPE. The present inventors used the CORASON program that generates a multi-locus, approximately maximum-likelihood, phylogenetic tree of BGCs including PKS families responsible for production of lichen substances. For the PKS23 family, an OMT (Crg06815) in C. rangiferina was used as the query gene to search for homologous BGCs from a total of 1,527 BGCs detected in the 30 sequenced species. For the PKS1 family, a GAL4-type transcription factor (gene ID: Crg07068) in C. rangiferina was used as the query gene.

### Example 3: Phylogenetic Analysis of Fungal NR-PKS

KS domain of iterative type I PKS has been considered evolutionarily conserved, and thus it can serve as a proxy for the similarity of the entire PKS. The present inventors identified a total of 242 PKSs from the genomes of the six Cladonia spp. and S. alpinum, among which four PKSs (Cgr01615, Cgr03964, Cgr08611, and Cmt10189; see Data Set S2) were missing a KS domain. KS domain sequences were extracted from 238 PKSs using the online tool NaPDoS and aligned using MUSCLE (v3.8.31). For clustering analysis, an all-versus-all similarity matrix for KS domains of 238 PKSs was computed using the AlignBuddy function in the BuddySuite program, with an optional argument: ‘-pi’. A heatmap showing percent similarity of KS domains clustered by k-means was generated using an R package Superheat. For fungal NR-PKS phylogeny, the present inventors used concatenated protein sequences of KS and PT domains of 103 NR-PKSs found in the six Cladonia spp. and S. alpinum, and 82 NR-PKSs that have been linked to known compounds in non-lichenized fungi. The present inventors initially identified 106 NR-PKSs in the six Cladonia spp. and S. alpinum, including seven NR-PKSs whose full sequences cannot be reliably defined from the current genome assembly (likely pseudogenes). Among these partial PKSs, three NR-PKSs (Cbo04702, Cma06590, and Cmt06606) lacked either KS or PT domain, and were excluded from the phylogenetic analysis. PT domains of lichen NR-PKSs were identified by aligning with those of previously characterized PKSs. A 6-methylsalicylic acid synthase (6MSAS) responsible for the biosynthesis of patulin (UniProtKB: A0A075TRC0) in Penicillium expansum was set to be an outgroup to fungal NR-PKS phylogeny. Also, four 6MSASs found in four Cladonia spp. were included in the analysis (Cbo07291, Cgr05254, Cmt10005, and Cuc03485). Protein sequences of KS and PT domains were aligned using MAFFT (v7.310) with the ‘auto’ setting, and spurious sequences or poorly aligned regions from each domain were trimmed using the trimAl program (v1.2), with the ‘gappyout’ parameter. The resulting multiple sequence alignments for KS and PT domains were concatenated with FASconCAT-G (v1.04). From the concatenated sequences, maximum-likelihood trees were computed with RA×ML (v8.2), using a gamma distribution for substitution rate across sites with a parameter setting: ‘-m PROTGAMMAWAG’. Nodal support was evaluated by 1,000 bootstrap replications. The final tree was rooted to the 6MSAS outgroup and annotated by iTOL (v5.7).

### Example 4: Generation of a “Clean Host” from Ascochyta rabiei

A split-marker strategy was employed to generate a solanapyrone-minus mutant (“clean host”). A downstream region of the sol1 (1,208 bp) and an upstream region of the sol3 (1,459 bp) were amplified from gDNA of As. rabiei isolate AR628 (39), using L5/L3 and R5/R3 primer pairs, respectively. A hygromycin phosphotransferase gene (hph) cassette was amplified from pCB1004 plasmid, using a primer pair HYG5/HYG3. L3 and R5 primers have 27 nt-long overhang sequences complementary to the 3′ and 5′ end of the hph cassette, respectively, such that the amplified sol1 downstream and sol3 upstream regions can be fused to the hph cassette by overlap extension PCR. Finally, split marker constructs were amplified from the fused construct, using N5/HY-R and YG-F/N3 primer pairs. The split marker constructs were introduced into protoplasts of As. rabiei by polyethylene glycol-mediated genetic transformation as described in Hallen-Adams et al. Primers used in the gene replacement were listed in Table 1.

Information on the primers used in the present invention is summarized in Table 1 below.

TABLE 1

| Pirmer | Sequence (5'-3') | No | Description |
|---|---|---|---|
| Sol1PpDS33 | AGATCTCGGCACGTTTTGATCGC | 7 | soll promoter |
| _fwd | AAAG | | cloning into |
| | | | pDS33 |
| Sol1PpDS33 | AAGCTTAGTAATATCTGGCGATG | 8 | sol1 promoter |
| _rev | AAGCGTCAG | | cloning into |
| | | | pDS33 |
| Inf_Sta046 | CAGATATTACTAAGCTCATGGCG | 9 | PKS23 cloning |
| 440_fwd | TCTAACCATGAGGAG | | into pDS35 |
| Inf_Sta046 | GTAACGTTAAGTGGATCCTTACA | 10 | PKS23 cloning |
| 440_rev | ACTAAAGCTCTCAATCAACCC | | into pDS35 |
| Sta046440 | GGACGATGAGAACGTCCTAG | 11 | PKS23 sequencing |
| check1F | | | |
| Sta046440 | CGAGTCAGGTGATCCAGTG | 12 | PKS23 sequencing |
| check2F | | | |
| Sta046440 | CCAATCATACTGAGCGCTG | 13 | PKS23 sequencing |
| check3F | | | |
| Sta046440 | CACATGTCTGTGGCTTGAG | 14 | PKS23 sequencing |
| check4F | | | |
| Sta046440 | CAACTCCGGTGATGATTG | 15 | PKS23 sequencing |
| check5F | | | |
| Sta046440 | CAACCCTTCCACATATTGAG | 16 | PKS23 sequencing |
| check6F | | | |
| pII99_sol5 | GAATTCAGAGCGACAGTGAAAGC | 17 | so15 promoter |
| _fwd | | | cloning into |
| | | | pII99 |
| pII99_sol5 | AGATCTAGCTAGCTTGGCGTGAT | 18 | so15 promoter |
| _rev | GTCAGTCAATGCTG | | cloning into |
| | | | pII99 (NheI |
| | | | enzyme site in |
| | | | bold) |
| pII99_tef1 | GAATTCCAGCCGAGACAGCAGAA | 19 | tefla promoter |
| a_fwd | TCAC | | cloning into |
| | | | pII99 |
| pII99_tef1 | AGATCTAGCTAGCGTGTTATGTT | 20 | tefla promoter |
| a_rev | TTGTGGAATATAAAAGGG | | cloning into |
| | | | pII99 (NheI |
| | | | enzyme site in |
| | | | bold) |
| Inf95_Sta0 | ATCACGCCAAGCTAGCACGATGA | 21 | atr3 cloning into |
| 4642_fwd | CTTCCGTTGATACAATG | | pII95 |
| Inf98_Sta0 | AAACATAACACGCTAGCACGATG | 22 | atr3 cloning into |
| 4642_fwd | ACTTCCGTTGATACAATG | | pII98 |
| Inf99_Sta0 | GGAGACCGGCAGATCCCGTCACA | 23 | atr3 cloning into |
| 4642_rev | TGTCCGTCATAATAAC | | pI195 and pII98 |
| Inf98_Sta0 | AAACATAACACGCTAGAACCATG | 24 | atr2 cloning into |
| 4643_fwd | GCTCTCCTAGACACAATTG | | pII95 |
| Inf95_Sta0 | ATCACGCCAAGCTAGAACCATGG | 25 | atr2 cloning into |
| 4643_fwd | CTCTCCTAGACACAATTG | | pII98 |
| Inf99_Sta0 | GGAGACCGGCAGATCGAGGAGCA | 26 | atr2 cloning into |
| 4643_rev | AAGGAACAAGGAACAG | | pI195 and pII98 |
| pII99_MK5 | CGTCAAGAGACCTACGAGACTG | 27 | Replacing nptII |
| | | | with ble in the |
| | | | plasmid |
| | | | teflα::atr2/pII98 |
| pII99_MK3 | GCTATACTTCTAGGTCTTGGAAG | 28 | Replacing nptII |
| | AG | | with ble in the |
| | | | plasmid |
| | | | teflα::atr2/pII98 |
| Inf_BLE_pI | GTAGGTCTCTTGACGCATTAAGA | 29 | Replacing nptII |
| I99_fwd | CCTCAGCGCTAGTGG | | with ble in the |
| | | | plasmid |
| | | | teflα::atr2/pII98 |
| Inf_BLE_pI | GACCTAGAAGTATAGCGGTGTTA | 30 | Replacing nptII |
| I99_rev | CGGAGCATTCACTAGG | | with ble in the |
| | | | plasmid |
| | | | teflα::atr2/pII98 |
| L5 | GAGGTCTTCACACCACAAGTTCG | 31 | Generating “clean |
| | | | host” |
| L3 | GGCAAAGGAATAGAGTAGATGCC | 32 | Generating “clean |
| | GACCGGGCTCTTGCGTAGTGGTA | | host” |
| | CAGATG | | |
| R5 | GTTGACCTCCACTAGCTCCAGCC | 33 | Generating “clean |
| | AAGCGAATAGGTAACAAAGCCAG | | host” |
| | CCGAG | | |
| R3 | GAGAATTGCGGCGCAGGATGTTC | 34 | Generating “clean |
| | | | host” |
| N5 | CTGACTTTCTTCTTGCAGCCCTG | 35 | Generating “clean |
| | C | | host” |
| N3 | CTAACGATGTCGTTAGTCGCCTT | 36 | Generating “clean |
| | G | | host” |
| HYG-F | GCTTGGCTGGAGCTAGTGGAG | 37 | Generating “clean |
| | | | host” |
| HYG-R | CGGTCGGCATCTACTCTATTCCT | 38 | Generating “clean |
| | T | | host” |
| YG-F | CGATGTAGGAGGGCGTGGATATG | 39 | Generating “clean |
| | TC | | host” |
| HY-R | TGTAGTGTATTGACCGATTCCTT | 40 | Generating “clean |
| | GCG | | host” |
| P1_fwd | GGACGATGAGAACGTCCTAG | 41 | RT-PCR analysis |
| P1_rev | GAGAGTTCCTCATGCTCGTC | 42 | RT-PCR analysis |
| P2_fwd | CATCGACGACACGGAGATACTG | 43 | RT-PCR analysis |
| P2_rev | GACCGTGAGATTCTTTGAAGAGG | 44 | RT-PCR analysis |
| Actin1_fwd | CAATGGTTCGGGTATGTGCAAG | 45 | RT-PCR analysis |
| Actin1_rev | GAAGAGCGAAACCCTCGTAGAT | 46 | RT-PCR analysis |

### Example 5: Heterologous Expression of the Atr1 and RT-PCR Analysis

For efficient expression of foreign genes in the “clean host”, the present inventors constructed expression vectors that carry either the sol1 gene promoter (pDS35), the sol5 gene promoter (pII95), or translation elongation factor 1 alpha gene promoter (pII98). Then, the atr1, atr2 and atr3 genes were individually cloned to the expression vectors. Details on expression vector construction and cloning procedures are described in Text S1. For heterologous expression of the atr1, the sol1::atr1/pDS35 plasmid (5-10 μg) was used to transform the “clean host”. Putative transformants were subcultured on potato dextrose agar (PDA; BD Biosciences) containing 100 μg/ml of nourseothricin sulfate (clonNAT; GoldBio). Two transformants, designated as Sta04644-T16 and Sta04644-T25, exhibited resistant to the selective agent, and were subcultured on PDA overlaid with a nylon membrane for RNA extraction. After two weeks of culture, mycelia growing on PDA were scraped off from the nylon membrane with a single-edge razor blade and were subjected to total RNA extraction. Total RNA was extracted from hyphae ground in liquid nitrogen, using TRIzol reagent (Thermo Fisher Scientific), according to the manufacturer's instruction, with additional extraction steps: one phenol (pH 4.6)-chloroform-isoamyl alcohol (25:24:1) extraction followed by one chloroform extraction step after the initial TRIzol-chloroform phase separation. RNA pellets were dissolved in 88 μL of nuclease-free water and subjected to genomic DNA digestion by DNase treatment (Qiagen). Then, RNA samples were concentrated using the RNA Clean & Concentrator (Zymo research). The present inventors confirmed expression and intron splicing of the atr1 with four introns in RT-PCR analysis. Two primer sets were designed: (i) to amplify a flanking region of the first intron located at the 5′ region of the atr1 coding sequence, and (ii) to amplify a region harboring the second, third and fourth (the last) introns at the 3′ region of the atr1 coding sequence. Two hundred nanogram of total RNA was reverse-transcribed, and the atr1 and actin1 (as a positive control) were amplified, using a OneStep RT-PCR kit (Qiagen). Primers used in RT-PCR analysis and plasmid constructions were listed in the above Table 1.

### Example 6: Generation of a Heterologous Host Producing Atranorin

For introduction of tailoring enzymes into a atr1-expressing strain (Sta04644-T25), the four resulting plasmids (sol5::atr2/pII95, tef1α::atr2/pII98, sol5::atr3/pII95, and tef1α::atr3/pII98) were individually transformed into the Sta04644-T25 strain, and putative transformants were subcultured on PDA containing 200 μg/ml of G418 disulfate (Sigma-Aldrich). Transformants resistant to G418 disulfate were selected for metabolite identification. A strain harboring both sol1::atr1/pDS35 and sol5::atr3/pII95 was used for the generation of an atranorin-producing strain, which exhibited the greatest production of compound 2(proatranorin), the immediate precursor of atranorin. Since a neomycin phosphotransferase II gene (nptII) cassette that confer resistance to G418 disulfate was already integrated into the strain producing compound 2, the present inventors replaced the nptII cassette in the tef1α::atr2/pII98 plasmid with a bleomycin resistant protein gene (ble), the tef1α::atr2/pII98 plasmid was amplified using a primer pair ‘Inf_BLE_pII99_fwd’ and ‘Inf_BLE_pII99_rev’, and the ble was amplified from pAC1750. Then, the two PCR products were fused, using the In-Fusion HD Cloning Kit (Takara). The resulting plasmid was transformed into the strain producing compound 2, and putative transformants were subcultured on PDA containing 200 μg/ml of Zeocin (a member of bleomycin family distributed by Thermo Fisher Scientific).

To select transformants that produce atranorin, the chemical profile of transformants resistant to Zeocin were compared to that of a S. alpinum voucher specimen (the Korea National Arboretum accession: KHL0017342) from which the genome-sequenced LFF had been originally isolated. Detailed methodologies for HPLC, LC-MS/MS, and NMR spectroscopic analyses for culture extracts and purified compounds were fully described in Examples 11 to 13.

### Example 7: Data Availability

This whole genome shotgun project has been deposited at DDBJ/ENA/GenBank under accessions, JAFEKC000000000, JAEUBB000000000, and JAEUBA000000000 (BioProject: PRJNA693578, PRJNA693575, and PRJNA693574) for C. borealis, Parmelia cf. squarrosa, and S. alpinum, respectively. Raw LC-MS/MS data were deposited at the MassIVE database (http://massive.ucsd.edu) under the accession no. MSV000087081).

### Example 8: Genome Sequencing and Annotation

LFF were isolated from C. borealis and S. alpinum collected from the King George Island, Antarctica (S62°13′51″ W58°46′32″) and Parmelia cf. squarrosa collected from Mt. Deogyu, South Korea (N35°51′24″ E127°44′54″). The LFF were cultured for 3-6 months on malt extract agar media (BD Biosciences). Genomic DNAs were extracted using a standard phenol/chloroform extraction method and were sequenced on an Illumina HiSeq2000 or NovaSeq6000 instrument, generating 150 nucleotide paired-end reads. Illumina adaptors were trimmed, read quality was assessed, and contigs were assembled at Macrogen Inc. (Seoul, South Korea). Details on genome assembly statistics, including previously sequenced genomes, were provided in Data Set S1 in the supplemental material. The genome assemblies retrieved from the NCBI database were annotated using GenSAS (v.6.0) annotation pipeline. Default settings were used unless otherwise noted. In brief, low complexity regions and repeats were masked using RepeatModeler (v1.0.11) and RepeatMasker (v4.0.7) (www.repeatmasker.org), setting the DNA source to ‘Fungi’. A masked consensus sequence was generated on which ab initio gene prediction was performed using the following tools: (i) Augustus (v3.3.1), selecting A. nidulans as a trained organism, (ii) GeneMarkES (v4.33), (iii) Genscan (v1.0), using a parameter setting for Human and other vertebrates, and (iv) GlimmerM (v2.5.1), selecting Aspergillus as a trained organism. For homology-based prediction, the NCBI reference transcript and protein databases for Fungi were searched, using (v) BLAST+ (v2.7.1) and (vi) DIAMOND (v0.9.22), respectively. For the consensus gene model prediction using EVidenceModeler (v06-25-2012), the above-mentioned standalone gene predictions were weighted as follows: (i)-five, (ii)-ten, (iii)-one, (iv)-one, (v)-five, and (vi)-five. The genome annotations were assessed with BUSCO (v5b) using the Ascomycota_odb9 data set.

### Example 9: Expression Vector Construction for PKS Expression

The pDS23 plasmid carries a nourseothricin acetyltransferase gene (nat) cassette as a selection marker and an enhanced green fluorescent protein (eGFP) under the control of the constitutive promoter of glyceraldehyde-3-phosphate dehydrogenase gene (G3PD) derived from Aspergillus nidulans. To replace the G3PD promoter with multiple cloning sites in pDS23, the plasmid was linearized by AvrII and HindIII restriction enzymes, and a synthetic DNA (57-bp long) harboring several restriction enzyme sites (NheI, AatII, SacI, PmeI, BglII, and SacII) was cloned between the AvrII and HindIII sites of the plasmid. The resulting plasmid was named pDS33. To generate an expression vector that carries a native promoter of As. rabiei, the 1,270-bp DNA fragment containing the sol1 (encoding solanapyrone synthase) gene promoter amplified from gDNA of As. rabiei isolate AR628 was cloned to pDS33 precut with BglII and HindIII, yielding the pDS35 plasmid. The coding sequence of atr1 (Sta04644; see Data Set S2 in the supplemental material) was amplified with a primer pair ‘Inf Sta046440 fwd’ and ‘Inf Sta046440 rev’ (7,829 bp) and cloned to the pDS35 linearized by HindIII and BamHI, in replace of eGFP, using the In-Fusion HD Cloning Kit (Takara), which resulted in the plasmid sol1::atr1/pDS35.

### Example 10: Expression Vector Construction for Tailoring Enzyme Expression

The pII99 plasmid carries a neomycin phosphotransferase II gene (nptII) cassette as a selection marker. To generate an expression vector that carries the promoter of the sol5 (encoding a Diels-Alderase that catalyzes the last step of solanapyrone biosynthesis), the 579-bp DNA fragment containing the sol5 promoter amplified from gDNA of As. rabiei AR628 strain was cloned to pII99 precut with EcoRI and BglII, yielding the pII95 plasmid. To introduce an additional restriction enzyme site, the revere primer for the sol5 promoter amplification was designed to have NheI site (see Table 1). The pII95 plasmid was digested with NheI and BglII, and the PCR products of the atr3 (Sta04642) or atr2 (Sta04643) amplified from gDNA of S. alpinum was fused to the precut plasmid, resulting in the plasmids, sol5::atr3/pII95 and sol5::atr2/pII95. Similarly, to generate an expression vector that carries the constitutive promoter of translation elongation factor 1 alpha gene (tef1α) derived from A. nidulans, the plasmid pII98 was generated by cloning the 880-bp DNA fragment containing the tef1α promoter amplified from the pAC1750 plasmid into pII99, and the coding sequences plus terminator regions of the atr3 and atr2 were individually fused into the pII98 precut with NheI and BglII, generating the plasmids, tef1α::atr3/pII98 and tef1α::atr2/pII98.

### Example 11: HPLC Analysis and Metabolite Purification

For metabolite extraction and profiling, transformants were grown on PDA for 2-3 weeks and the whole agar containing fungal colony was excised from the Petri dish and soaked into 10 ml of ethyl acetate in a 50 ml falcon tube. After sonication for 30 min, an aliquot of 1 ml of solution was transferred to a 1.5 ml microcentrifuge tube, evaporated to dryness, reconstituted with 100 μl of methanol, and subjected to a high-performance liquid chromatography (HPLC) analysis. Pieces of the S. alpinum voucher specimen (20-30 mg) were soaked into 300 μl of acetone in a 2 ml microcentrifuge tube. After sonication for 30 min, the sample was filtered through 0.4 mm syringe membrane before injection to HPLC. Chemical profiling of transformants was performed using a Prominence Modular LC-20A HPLC instrument (Shimadzu). The samples were analyzed using a YMC-Pack ODS-A (column size, 150×4.6 mm; particle size, 5 μm; pore size, 12 nm; at 40° C.) and using a photodiode array detector at 254 nm (SPD-M20A, range 180-700 nm). The mobile phase was composed of distilled water/trifluoroacetic acid (99.9:0.1, v/v) for pump A and methanol/trifluoroacetic acid (99.9:0.1, v/v) for pump B. The HPLC analysis was performed using a gradient program at a flow rate of 1.0 ml/min: 0-30 min, 20-100%; 30-40 min, 100%; 40-52 min, 20% of pump B. The present inventors used the lichen substance database for HPLC analysis to identify metabolites found in lichen specimens (15). For structural identification, the present inventors purified compounds 1, 2, and 4 from culture extracts of transformants producing the compounds as major metabolites. Twenty PDA cultures were extracted three times with ethyl acetate. Culture extracts were evaporated to dryness on a Rotavapor (Buchi), reconstituted with methanol, and subjected to preparative HPLC. Separation was achieved on a HPLC at a flow rate of 2.2 ml/min, using the Kromasil C18-column (250×10 mm, 5 μm, 10 nm; at 40° C.), with UV monitoring at 254 nm. The compound 1 was purified using 66% acetonitrile (tR=9.86 min; 9.5 mg); compound 2 was purified using 85% acetonitrile (t_{R}=9.77 min; 6.2 mg); compound 4 was purified using 80% acetonitrile (tR=9.60 min; 2.1 mg). Solvents were buffered with 0.1% trifluoroacetic acid. The purified compounds were subjected to NMR spectroscopic analyses.

### Example 12: LC-MS/MS Analysis for Identifying Products

Culture extracts were 20-folds diluted with methanol and filtered with a 0.22 μm PTFE filter. The LC-MS analysis was performed on a Waters Acquity I-Class UPLC system coupled to a Waters VION IMS QTOF mass spectrometer (Waters Co.), which was equipped with an electrospray ionization interface. The mobile phase was comprised of 0.1% formic acid in water (pump A) and acetonitrile (pump B). A stepwise gradient method at a constant flow rate of 0.3 ml/min was used to elute the column with the following conditions: 20-100% of pump B (0-9 min), followed by 3 min of washing and 3 min of reconditioning. The injection volume was 2 μl. The column temperature and sample organizer were maintained at 40° C. and 20° C., respectively. Tandem MS analyses were performed in data-independent acquisition (MS^{E}), negative ion mode with the m/z 50-1500 Da range and acquisition times of 0.2 s. The low collision energy for the detection of the precursor ions was set to 6 eV, while the high collision energy for fragmentation was set to 20-40 eV. The ionization conditions were set as follows: the capillary voltage was 2.5 kV, the cone voltage was 40 V, the source temperature was 100° C., the desolvation temperature was 250° C., the cone gas flow was 0 l/h, and the desolvation gas flow was 800 l/h. High-purity nitrogen was used as the nebulizer and auxiliary gas, and argon was used as the collision gas. The [M-H]⁻ ion of leucine enkephalin at m/z 554.2615 was used as the lock mass to ensure mass accuracy and reproducibility. MS/MS spectral data matching of major compounds detected in transformants was performed by the feature-based molecular networking workflow in GNPS after the preprocessing using MS-DIAL.

### Example 13: NMR Spectroscopic Data Summary

13-1: 4-O-demethylbarbatic acid (Compound 1)

¹H NMR (400 MHz, CD₃COCD₃): δ_{H} 11.65 (s, OH), 6.73 (s, 1H), 6.47 (s, 1H), 2.68 (s, 3H), 2.63 (s, 3H), 2.12 ( [symbol] s, 3H×2); ¹³C NMR (100 MHz, CD₃COCD₃): δ_{C} 170.4, 164.0, 163.3, 160.6, 153.0, 140.9, 140.7, 116.7, 116.6, 111.3, 109.6, 109.2, 103.6, 23.8, 23.1, 8.6, 7.3; ¹³C NMR signals for carboxyl group were not observed or very-low-intensity maybe owing to the long relaxation times of these carbon nuclei, δ_{C} of carboxyl group was 174.1 in published data. The 1H NMR and ¹³C NMR data were comparable to published data. HRESIMS m/z 345.0972 [M-H]⁻ (calcd. for C₁₈H₁₇O₇, 345.0974); the MS/MS spectrum is deposited in the GNPS spectral library.

### 13-2: Proatranorin I (Compound 2)

¹H NMR (400 MHz, CDCl₃): δ_{H} 11.90 (s, OH), 11.70 (s, OH), 6.50 (s, 1H), 6.29 (s, 1H), 3.96 (s, 3H), 2.60 (s, 3H), 2.52 (s, 3H), 2.12 (s, 3H), 2.07 (s, 3H); ¹³C NMR (100 MHz, CDCl₃): δ_{C} 172.4, 170.3, 164.2, 162.9, 159.0, 152.6, 140.7, 139.8, 117.0, 116.4, 111.2, 110.1, 109.0, 104.2, 52.3, 24.6, 24.1, 9.4, 7.7; 1H NMR (400 MHz, CDCl₃) data was comparable to published data; ¹³C NMR (100 MHz, CDCl₃) data was comparable to 4-O-demethylbarbatic acid (Compound 1) data, except that there was one more oxygen-methyl carbon (δ_{C} 52.3) in compound 2. HRESIMS m/z 359.1132 [M-H]⁻ (calcd. for C₁₉H₁₉O₇, 359.1131); the MS/MS spectrum is deposited in the GNPS spectral library.

### 13-3: Proatranorin III (Compound 4)

¹H NMR (400 MHz, CDCl₃): δ_{H} 12.54 (s, OH), 12.45 (s, OH), 11.77(s, OH), 10.35(s, CHO), 6.54 (s, 1H), 6.40 (s, 1H), 2.68 (s, 3H), 2.59 (s, 3H), 2.08 (s, 3H); 1H NMR (400 MHz, CDCl₃) data was comparable to published data of atranorin (Compound 5), except that there was missing a methyl group (δ_{H} 3.99) at the carboxyl group in (Compound 4). HRESIMS m/z 359.0771 [M-H]⁻ (calcd. for C18H15O8, 359.0767); the MS/MS spectrum is deposited in the GNPS spectral library.

### Example 14: PKS Families in the Genus Cladonia

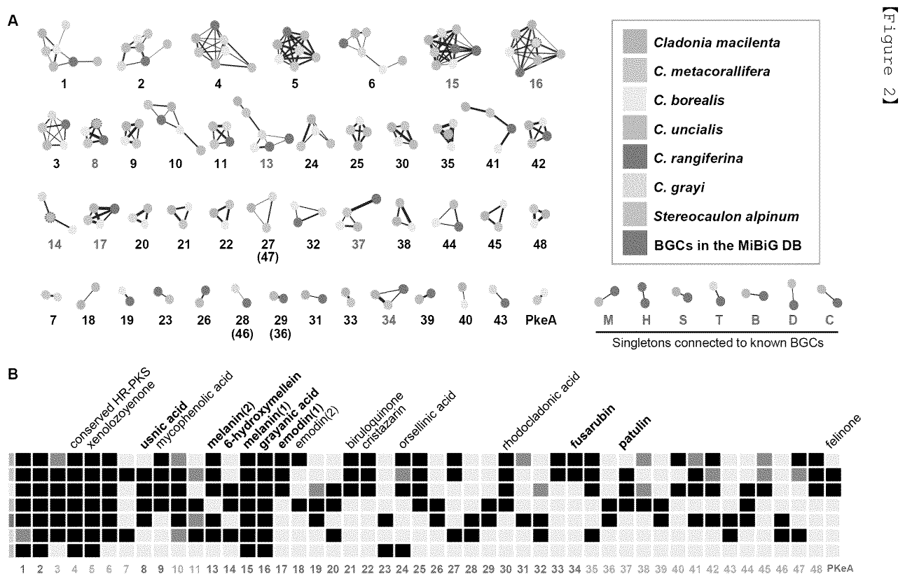

Forty-five gene cluster families (GCFs) including at least one iterative type I PKS gene were identified, using the Big-SCAPE program. Biosynthetic gene clusters (BGCs) of PKS1, PKS2, PKS4, PKS5, PKS15 and PKS16 families were conserved in the six Cladonia species plus S. alpinum, and BGCs of PKS3 and PKS10 families were conserved in the six Cladonia species. Previously, Timsina et al. identified thirteen PKS families conserved in species in the Cladonia chlorophaea species complex (PKS1, PKS2, PKS3, PKS5, PKS7, PKS10, PKS11, PKS12, PKS13, PKS14, PKS15, PKS16 and MSAS). The present inventors also identified these PKS families in our gene cluster network analysis (FIG. 2A). However, the present inventors failed to detect PKS12 homologs in the genomes of the six Cladonia species, even in the C. grayi genome from which the PKS12 family was originally identified. Three PKSs are manually detected and linked to respective PKS families, as the gene annotations were incomplete and fragmented so that the antiSMASH program failed to detect BGCs harboring the three PKSs: Cma06514 (PKS3), Cgr07347 (PKS10), and Cuc08442 (PKS19).

### Example 15: Genetic Dereplication of Cladonia BGCs (Biosynthetic Gene Clusters)

To link unknown biosynthetic gene clusters in the six Cladonia spp. and S. alpinum to known compounds in other fungi, GCFs predicted by the Big-SCAPE program were coupled to previously characterized BGCs deposited in the MIBiG (Minimal Information about a Biosynthetic Gene cluster) database (v.1.4). As a result, eight GCFs and seven singleton BGCs were linked to known BGCs found in non-lichenized fungi (FIG. 2A). These conserved biosynthetic gene clusters (BGCs) in phylogenetically distant species suggested shared ancestries of the biosynthetic pathways between lichens and non-lichenized fungi. Nevertheless, these conserved BGCs exhibited a high degree of gene content variation, suggesting divergent pathways that may have led to chemical diversity. There were three lichen BGCs listed in the MIBiG database: BGCs for PKS8 in C. uncialis, PKS14 in C. uncialis, and PKS16 in C. grayi, which were indicated by dashed red circles in FIG. 2A.

### Example 15-1: PKS8

The PKS8, also known as methylphloroacetophenone synthase (MPAS) in C. uncialis, is known to be involved in biosynthesis of usnic acid, based on phylogenetic analysis and gene expression study. A GCF of the PKS8/MPAS family is shared by C. borealis, C. metacorallifera, C. rangiferina, and C. uncialis. However, among these four Cladonia species, usnic acid has not been detected in C. rangiferina.

### Example 15-2: PKS14

The putative 6-hydroxymellein BGC found in C. uncialis (BGC0001489) formed a GCF with PKS14 BGCs in C. borealis and C. grayi. The roles of the gene members of the 6-hydroxymellein BGC were tentatively assigned based on the terrein biosynthetic pathway, in which terA and terB act in concert to biosynthesize 6-hydroxymellein in A. terreus. In the present inventor's network analysis, the terrein BGC (MIBiG accession: BGC0000161) was not linked to the 6-hydroxymellein BGCs in the three Cladonia species, likely due to gene content variation observed between them; only three genes (terA, terB and terD) among the eleven gene members of the terrein BGC in A. terreus were conserved in the 6-hydroxymellein BGCs in the three Cladonia species.

### Example 15-3: PKS13 PKS15

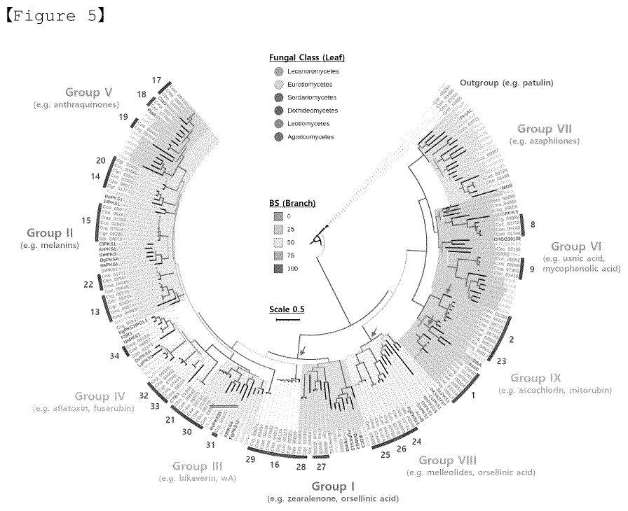

BGCs of the PKS15 family conserved in the six Cladonia spp. and S. alpinum were linked to many previously characterized BGCs including a 1,3,6,8-tetrahydroxynaphthalene (T4HN) synthase responsible for melanin production in diverse fungi. For brevity, FIG. 2A showed only one BGC containing a T4HN synthase (BGC0001257) connected to the BGCs of the PKS15 family. The present inventors identified that PKS13 BGCs were also connected to known BGCs containing a T4HN synthase. Unlike the PKS15 family conserved in the six Cladonia spp. and S. alpinum, the PKS13 family was not found in C. uncialis and S. alpinum, showing 51-54% protein sequence identity with ElcPKS1 responsible for the melanin production in Exophiala lecanii-corni (FIG. 5). The ElcPKS1 and PKS13 family formed a highly supported clade (bootstrap value of 99%) distinct from the PKS15 family and other conventional T4HNs, indicating that some Cladonia species possess two copies of melanin PKSs of different origin.

### Example 15-4: PKS16

The putative grayanic BGC found in C. grayi (BGC0001266) is conserved in the six Cladonia spp. and S. alpinum. The PKS16 BGC was proposed to produce orsellinic acid-derived grayanic acid (a depsidone) that is found in C. grayi.

### Example 15-5: PKS17

A GCF of the PKS17 family, shared by C. borealis, C. macilenta and C. metacorallifera, was linked to a BGC (BGC0001583) for the biosynthesis of emodin, an anthraquinone produced by many different fungi. In addition to the four core enzymes (an NR-PKS, metallo-β-lactamase, decarboxylase and anthrone oxygenase) that are necessary and sufficient for emodin biosynthesis, a few more tailoring enzymes including a cytochrome P450 monooxygenase were found in the homologous Cladonia BGCs. The P450 monooxygenase showed 39% sequence identity with the ClaM that mediates the coupling of two emodin derivatives in Cladosporium fulvum, suggesting that the P450 enzyme may be involved in biosynthesis of skyrin. Intriguingly, another BGC harboring the four core genes for emodin biosynthesis was found in the genome of C. macilenta, although in different arrangement with the PKS17 BGCs. This second BGC inC. macilenta for putative emodin biosynthesis formed a GCF with the PKS18 BGC in C. uncialis, which was homologous to the BGC of pestheic acid, an emodin derivative, found in Pestalotiopsis fici.

### Example 15-6: PKS34

A GCF of the PKS34 family shared byC. macilenta and C. metacorallifera was linked to a BGC (BGC0001242) for the biosynthesis of fusarubin, the red mycelial pigment produced by Fusarium fujikuroi. However, the BGC contents were highly variable; only putative homologs of fsr1 (encoding an NR-PKS), fsr4 (encoding an alcohol dehydrogenase) and fsr5 (encoding a short-chain dehydrogenase) in the fusarubin BGC can be found in the PKS34 BGCs.

### Example 15-7: PKS37

The patulin BGC (BGC0000120) in Penicillium expansum was connected to a GCF of the PKS37 family shared by C. borealis, C. metacorallifera and C. uncialis. Among the 15 gene members of the patulin BGC, ten genes homologous to patF-patO can be found in the three Cladonia spp. (except for patF in C. borealis), which are necessary for the biosynthesis of neopatulin, the immediate precursor of patulin. Homologs of patD and patE responsible for the final conversion of neopatulin to patulin were absent in the Cladonia genomes. Instead, other tailoring enzymes found were an epimerase in C. metacorallifera and C. uncialis, a TauD-like dioxygenase in C. borealis and C. uncialis, and two short chain dehydrogenases in C. uncialis.

### BGC Example 16: Singleton BGCs Connected to the MIBiG Database

Several previously characterized BGCs were connected to BGCs found in a single species (singleton BGCs). These singleton BGCs tend to exhibit little gene content variation and high sequence similarity to corresponding BGCs found in non-lichenized fungi, suggesting recent introductions of these BGCs into lichens. These likely horizontally transferred BGCs from non-lichenized fungi were as follows: the sorbicillin BGC (BGC0001404) linked to a BGC inC. macilenta, the terreic acid BGC (BGC0000160) linked to a BGC in C. grayi, the hypothemycin BGC (BGC0000076) linked to a BGC in C. rangiferina, the curvupallides BGC (BGC0001563) linked to a BGC in C. uncialis, and the depudecin BGC (BGC0000046) linked to a BGC in S. alpinum. The monascorubrin BGC (BGC0000099) and betaenones BGC (BGC0001264) were also connected to BGCs in C. uncialis in the present inventors' network analysis, as well as in an earlier study on homology mapping of C. uncialis PKSs.

### Example 17: Notes on Homology Mapping of Cladonia PKS Families

### Example 17-1: Homology Mapping of Conserved Lichen PKS Families

Although gene cluster network analysis ascribed many lichen BGCs to known compounds with high likelihood, there may be more homologous BGCs that were not connected to previously characterized BGCs in non-lichenized fungi, due to high gene content variations. Therefore, the present inventors performed BLAST searches of 45 PKS families against the NCBI non-redundant protein sequences database to identify Cladonia PKS families homologous to PKS genes with known compounds.

### Example 17-2: PKS4

The PKS4 family encodes an R-PKS enzyme conserved in the six Cladonia spp. and S. alpinum. The PKS4 family can be found in lichenized and non-lichenized fungi and appears to have undergone co-evolution with the physically-associated type III PKS.

### Example 17-3: PKS5

The PKS5 family was conserved in the six Cladonia spp. and S. alpinum, encoding a PKS-NRPS hybrid enzyme that showed a high sequence similarity to the xenolozoyenone PKS in Glarea lozoyensis (39) (56% at protein sequence level). Homologs of the PKS5 family can be found in diverse fungi (39). Interestingly, two tandem genes encoding R-PKS and NRPS enzymes for the xenolozoyenone biosynthesis in G. lozoyensis are co-transcribed into one dicistronic mRNA under the control of the same promoter. Thus, it needs to be examined whether the PKS5 family in lichens also encodes two separate polypeptides for R-PKS and NRPS enzymes or a single peptide for a PKS-NRPS hybrid enzyme.

### Example 17-4: PKS9

The PKS9 family was shared by C. borealis, C. macilenta, C. metacorallifera and C. uncialis, which is similar to an NR-PKS (MpaC) responsible for the biosynthesis of mycophenolic acid derived from 5-methylorsellinic acid in Penicillium brevicompactum (44% protein sequence identity), as described in a previous homology mapping study. Also, the PKS9 family showed 43-45% protein sequence identity to NR-PKSs (AdrD, AndM, AusA, NvfA, PrhL and Trt4) involved in the biosynthesis of meroterpenoids derived from 3,5-dimethylorsellinic acid in Aspergillus and Penicillium species. However, the PKS9 BGCs in the Cladonia genomes lacked biosynthetic genes related to terpene biosynthesis, such as prenyltransferase and terpene cyclase.

### Example 17-5: PKS18

The PKS18 family shared by inC. macilenta and C. uncialis showed 64-68% protein sequence identity to an NR-PKS (PtaA) involved in the biosynthesis of pestheic acid, an emodin derivative, in Pestalotiopsis fici, as described in a previous homology mapping study.

### Example 17-6: PKS21

A purple pigment biruloquinone was first discovered in a foliose lichen Parmelia birulae. Although biruloquinone has not been found in Cladonia species in nature, the present inventors previously identified a chemical variant of the genome-sequenced mycobiont isolated from C. macilenta, producing biruloquinone in axenic culture. The present inventors tentatively assigned the PKS21 family to the biosynthesis of biruloquinone by the whole-transcriptome comparison between the biruloquinone producer and non-producer.

### Example 17-7: PKS22

A red pigment cristazarin, a naphthazarin derivative, was originally identified from a mycobiont culture of C. cristatella. The present inventors established an optimum culture condition for cristazarin production in a mycobiont isolated from the genome-sequenced C. metacorallifera and ascribed the PKS22 family to the biosynthesis of cristazarin, based on the observation that the PKS23 was highly upregulated in cristazarin-inducing conditions in C. metacorallifera.

### Example 17-8: PKS24

The PKS24 family was shared by C. borealis, C. macilenta, C. metacorallifera and S. alpinum. The PKS24 BGC in C. metacorallifera was slightly different from the ones in the other three species, with a fragmented PKS24 gene. The PKS24 family formed a monophyletic clade maximally supported by 100% bootstrap with FgPKS14 involved in orsellinic acid biosynthesis in Fusarium graminearum, showing 46-48% protein sequence identity to FgPKS14 (FIG. 5).

### Example 17-9: PKS30

The PKS30 family showed 52-53% protein sequence identity to an NR-PKS (wA) responsible for the biosynthesis of the conidial yellow pigment (aka. YWA1), a naphthopyrone produced by A. nidulans. The PKS30 BGCs conserved in C. borealis, C. macilenta, C. metacorallifera and C. uncialis are likely involved in the biosynthesis of the red apothecial pigment, rhodocladonic acid, albeit the pigment has not been detected in C. uncialis. There were only two GCFs (the PKS21 and PKS30 families) shared by the three red apothecial pigment producers, C. borealis, C. macilenta and C. metacorallifera. The PKS21 and PKS30 families belong to the NR-PKS Groups III and IV, respectively, which include NR-PKSs involved in the biosynthesis of naphthopyrone-derived compounds (FIG. 5). Since the PKS21 family was tentatively assigned to the biosynthesis of biruloquinone (a phenanthrenequinone), the PKS30 family is the last remaining naphthopyrone PKSs shared by the three red apothecial pigment producers. This putative assignment was strengthened by the presence of enzymes necessary for the biosynthesis of rhodocladonic acid, such as O-methyltransferase and two monooxygenases in the PKS30 BGCs. A proposed biosynthetic pathway for rhodocladonic acid from a naphthopyrone precursor was shown below.

### Example 17-10: PkeA

BGCs shared by C. borealis and C. metacorallifera included an NR-PKS showing 63-65% protein sequence identity to PKeA (also known as DbaI) responsible for the biosynthesis of felinones in A. nidulans. The present inventors refrained from assigning a PKS family number for the NR-PKSs homologous to PkeA/DbaI, as these PKS genes appear to have arisen through horizontal transfer and may be specific to a certain lineage of Cladonia, as with the other Cladonia NR-PKSs that belong to the Group VII (e.g., sorbicillin BGC in C. macilenta).

### Example 18: Metabolic Potentials of Lichen

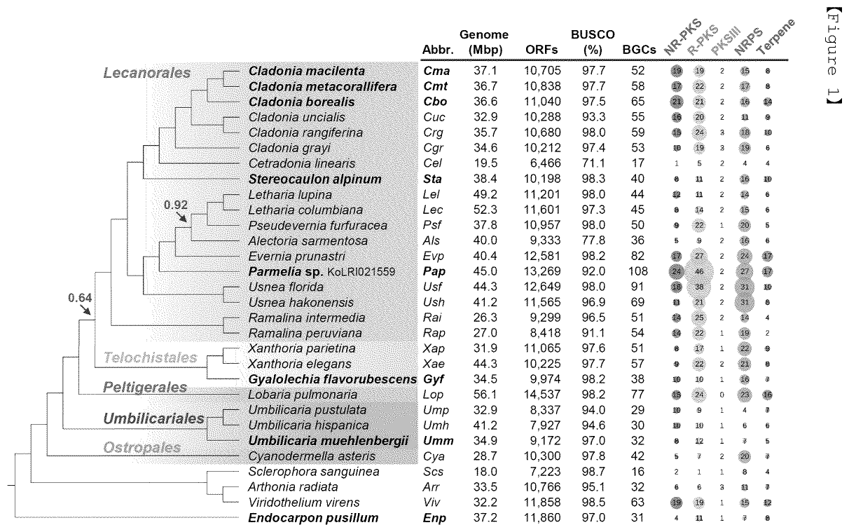

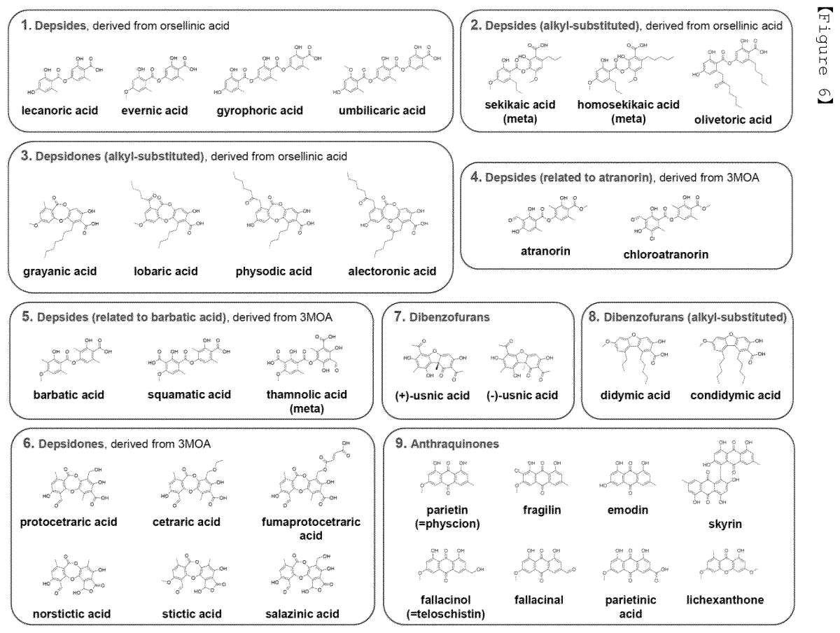

To examine evolutionary relationships of LFF and compare their genome-encoded metabolic potentials, a coalescent-based tree of 393 single-copy orthologous genes was reconstructed, using eight genomes that we have sequenced and 22 genome assemblies available in the NCBI database and JGI website, as of September 2020. A majority of the sequenced species (26 out of 30) belong to the Lecanoromycetes and four LFF were placed outside of the Lecanoromycetes: Arthonia radiata (Arthoniomycetes), Endocarpon pusillum (Eurotiomycetes), Sclerophora sanguinea (Coniocybomycetes) and Viridothelium virens (Dothideomycetes). Barring the genomes of Alectoria sarmentosa and Cetradonia linearis, completeness of the genomes assessed by BUSCO analysis was greater than 91% (FIG. 1). An endangered lichen Ce. linearis and Sc. sanguinea had compact genomes shorter than 20 Mbp (FIG. 1). Except for these two species, all genomes were 26.3-56.1 Mbp in size, containing 7, 927-14,537 open reading frames (FIG. 1). Genome-encoded metabolic potentials of the 30 species were investigated by mining the genomes for BGCs using antiSMASH. Metabolic potentials of the sequenced LFF were highly variable, with BGC counts of 16-108 (FIG. 1); Parmelia sp. KoLRI021559 showed the greatest BGC diversity, whereas the genomes of Ce. linearis and Sc. sanguinea carried the smallest sets of BGCs which may be attributable to their contracted genome size. Notably, the six Cladonia genomes possess 52-65 BGCs and encode similar numbers of PKSs; an average of 16 NR-PKSs and 20 reducing type PKS (R-PKS) (FIG. 1). The six Cladonia spp. produce unique sets of SMs of polyketide origin (see Table 2 and FIG. 6), which make it ideal for studying inter-species PKS gene diversity and identifying BGCs responsible for biosynthesis of cortical or medullary substances of lichens through comparative analysis.

Table 2 below summarizes the lichen materials reported in the genome-sequence analysis of 30 species.

TABLE 2

| speices | Lichen substances |
|---|---|
| Cladonia macilenta | barbatic acid, didymic acid |
| Cladonia metacorallifera | thamnolic acid), usnic acid, didymic acid |
| Cladonia borealis | barbatic acid, usnic acid |
| Cladonia uncialis | squamatic acid, usnic acid |
| Cladonia rangiferina | atranorin, fumaprotocetraric acid |
| Cladonia grayi | grayanic acid, fumaprotocetraric acid |
| Cetradonia linearis | — |
| Stereocaulon alpinum | lobaric acid, atranorin, stictic acid |
| Letharia lupina | atranorin, norstictic acid |
| Letharia columbiana | atranorin |
| Pseudevernia furfuracea | olivetoric acid, physodic acid, atranorin/ |
| | chloroatranorin |
| Alectoria sarmentosa | alectoronic acid, usnic acid |
| Evernia prunastri | lecanoric acid/evernic acid, physodic acid, |
| | atranorin/chloroatranorin, salazinic acid, |
| | usnic acid |
| Parmelia sp. | atranorin/chloroatranorin, salazinic acid |
| KoRLI021559 | |
| Usnea florida | thamnolic acid, norstictic acid/salazinic acid, |
| | usnic acid |
| Usnea hakonensis | usnic acid |
| Ramalina intermedia | sekikaic acid, atranorin, usnic acid |
| Ramalina peruviana | sekikaic acid, usnic acid |
| Xanthoria parietina | parietin/fallacinol/parietinic acid/emodin |
| Xanthoria elegans | parietin |
| Gyalolechia flavorubescens | parietin/fallacinol/fragilin/emodin |
| Lobaria pulmonaria | gyrophoric acid, atranorin, norstictic acid/ |
| | salazinic acid |
| Umbilicaria pustulata | lecanoric acid/gyrophoric acid/umbilicaric |
| | acid, skyrin |
| Umbilicaria hispanica | lecanoric acid/gyrophoric acid/umbilicaric |
| | acid, skyrin |
| Umbilicaria muehlenbergii | lecanoric acid/gyrophoric acid |
| Cyanodermella asteris | skyrin |
| Sclerophora sanguinea | — |
| Arthonia radiata | Not detected |
| Viridothelium virens | lichexanthone |
| Endocarpon pusillum | — |

### Example 19: Genetic Dereplication of Cladonia PKSs

To search for PKS genes involved in biosynthesis of lichen polyketides, namely depside and depsidone series compounds, the present inventors focused on 226 BGCs harboring at least one PKS in the six Cladonia spp. plus a related Antarctic lichen S. alpinum, from which a total of 242 PKSs were identified. The present inventors first conducted clustering analysis of the conserved ketosynthase (KS) domains of each PKS and identified a number of clusters that are suggestive of homologous relationships. Twelve PKS families described in an earlier study on the Cladonia chlorophaea species complex were also detected in our clustering analysis, indicating that a significant proportion of PKS genes are conserved in the genus Cladonia. However, little is known about their products. Therefore, the present inventors linked BGCs to known compounds in non-lichenized fungi, using the Big-SCAPE program that maps BGC diversity onto sequence similarity networks. The network analysis was used to graphically summarize three attributes of BGCs: (i) PKS families associated with the network (numbers), (ii) species distribution across the network (nodes), and (iii) degrees of similarity between pairs of BGCs (edges) (FIG. 2A). As depicted by the gene cluster network, the BGCs harboring at least one PKS gene were grouped into 45 gene cluster families (GCFs). Eight GCFs and seven species-specific BGCs (labelled in red) were coupled to previously characterized BGCs deposited in the MIBiG database. FIG. 2B summarized phyletic distribution of Cladonia PKS families, denoting signature SMs linked to Cladonia PKS families.

### Example 20: Identification of Putative Atranorin BGC

The gene cluster network analysis in the six Cladonia spp. and S. alpinum revealed that the PKS23 family among the 45 GCFs may be involved in biosynthesis of atranorin, the major cortical substance of diverse macrolichens, as it was the sole GCF shared by the two atranorin producers, C. rangiferina and S. alpinum (FIG. 2). Atranorin is unique in its structure having a methoxycarbonyl group within the 3MOA moiety, which is rarely found in other depsides and depsidones series compounds. A BLAST search with an O-methyltransferase (OMT) in the PKS23 BGC in C. rangiferina showed 37% protein sequence identity to the Trt5 (UniProtKB:Q0C8A3), an OMT that mediate the formation of a methoxycarbonyl group within the 3,5-dimethylorsellinic acid moiety in A. terreus during the biosynthesis of terretonin (a meroterpenoid) (Table 3).

Table 3 below summarizes the information on the PKS23 biosynthetic gene cluster of Cladonia rangiferina.

TABLE 3

| | | | Iden- | | |
|---|---|---|---|---|---|
| ORFa | Size | BLASTP | tity | Conserved | E- |
| (Crg) | (aa) | homolog^{b} | (%) | domain | value |
| 06811 | 744 | MFS mono- | 71 | Sugar transporter | 1e−105 |
| | | saccharide | | (Pfam00083) | |
| | | transporter | | | |
| 06812 | 306 | export control | 68 | chitin synthase | 1e−131 |
| | | protein) CHS7- | | III catalytic | |
| | | like | | subunit | |
| | | | | (Pfam12271) | |
| 06813 | 495 | TFIIH complex | 58 | TFIIH complex | 1e−82 |
| | | p47 subunit | | subunit Ssl1-like | |
| | | | | (Pfam04056) | |
| 06814 | 549 | MES multidrug | 50 | major facilitator | 9e−31 |
| | | transporter | | superfamily | |
| | | (atr4) | | (Pfam07690) | |
| 06815 | 346 | Trt5, O- | 37 | SAM- dependent | 1e−04 |
| | | methyl- | | methyltransferase | |
| | | transferase | | (Pfam08241) | |
| | | (atr3) | | | |
| 06816 | 508 | Cytochrome | 43 | Cytochrome P450 | 3e−57 |
| | | P450 | | (Pfam00067) | |
| | | monooxygenase | | | |
| | | (atr2) | | | |
| 06817 | 2,513 | non-reducing | 44 | SAT-KS-AT-PT- | |
| | | polyketide | | ACP-MT-TE | |
| | | synthase (atr1) | | | |
| 06818 | 472 | hypothetical | 33 | phospho- | 4e−08 |
| | | protein | | transferase | |
| | | | | enzyme family | |
| | | | | (Pfam01636) | |
| 06819 | 2,377 | reducing | 40 | KS-AT-DH-ER- | |
| | | polyketide | | KR-ACP | |
| | | synthase (R-PKS) | | | |
| 06820 | 184 | hypothetical | 40 | domain of | 5e−22 |
| | | protein | | unknown | |
| | | | | function, | |
| | | | | DUF3237 | |
| | | | | (Pfam11578) | |
| 06821 | 301 | hypothetical | 34 | domain of | 2e−09 |
| | | protein | | unknown | |
| | | | | function, | |
| | | | | DUF2306 | |
| | | | | (Pfam10067) | |
| 06822 | 236 | hypothetical | 28 | (not detected) | |
| | | protein | | | |
| 06823 | 356 | hypothetical | 32 | (not detected) | |
| | | protein | | | |
| 06824 | 565 | putative | 41 | flavin-binding | 3e−14 |
| | | dimethylaniline | | monooxygenase- | |
| | | monooxygenase | | like (Pfam00743) | |

aORF: Open reading frame of C. rangiferina); the synthetic core genes are highlighted in.
^{b}For BLAST searches, the NCBI non-redundant protein sequence database for four Aspergillus species is used: Aspergillus fumigatus (taxid: 746128), A. nidulans (taxid: 162425), A. niger (taxid: 5061), and A. terreus (taxid: 33178).

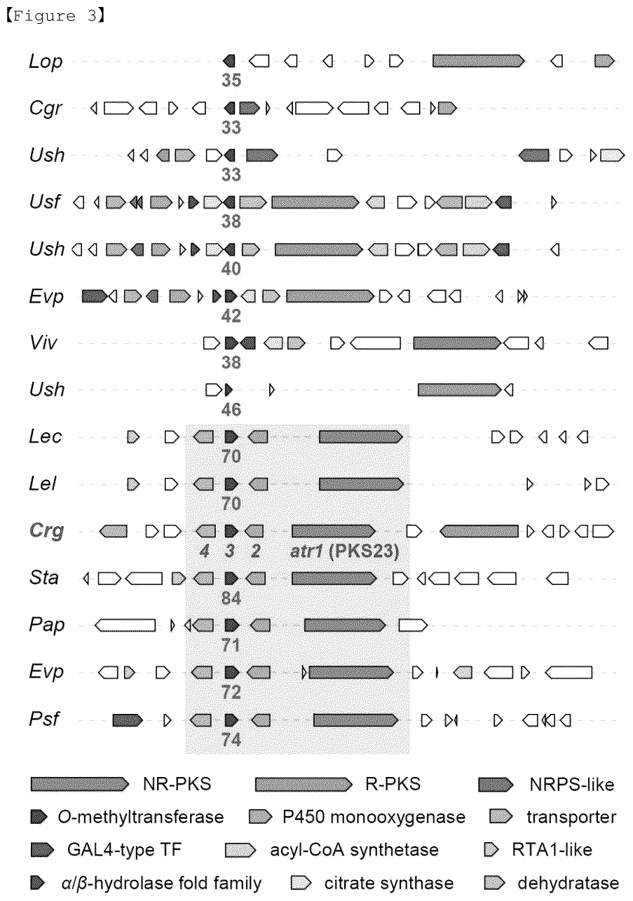

To examine presence of conversed PKS23 BGCs in other lichens that produce atranorin, the present inventors adopted the CORASON pipeline that is useful for studying conservation and variation of BGCs within and across GCFs. The protein sequence of the OMT in the PKS23 BGC in C. rangiferina was queried against a total of 1,527 BGCs detected in the 30 genomes. The present inventors identified 15 BGCs each harboring an OMT that showed a significant hit (>33% protein sequence identity) (FIG. 3). Among the 15 BGCs, seven BGCs contained four synthetic core genes: PKS23 (coined atr1), a cytochrome P450 monooxygenase (atr2), an OMT (atr3) and a transporter (atr4) (FIG. 3 and Table 1). These syntenic BGCs were exclusively found in the atranorin-producing lichens among the 30 sequenced species (see Table 2).

### Example 21: Reconstruction of Atranorin Biosynthetic Pathway

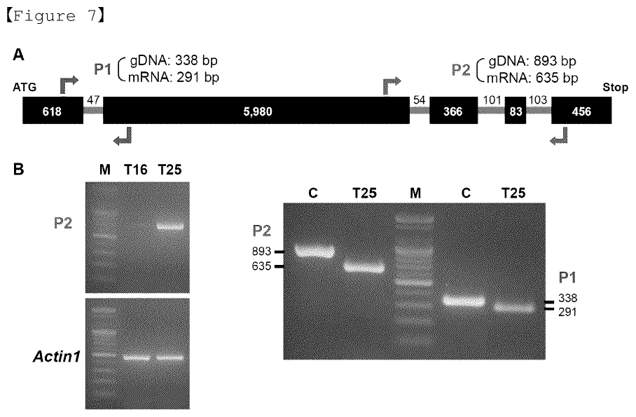

Despite arduous efforts made for heterologous expression of lichen PKS genes in well-established heterologous hosts, such as A. nidulans and A. oryzae, the attempts were unsuccessful for yet-unknown reasons (11, 23). Thus, the present inventors set out to establish a new heterologous expression system, using a plant pathogenic fungus Ascochyta rabiei (Dothideomycetes). On the basis of the chemical structure and the predicted functions of three biosynthetic genes in putative atranorin BGCs, a polyketide pathway for atranorin can be envisioned (FIG. 4A). To investigate the roles of individual genes, the present inventors first generated a “clean host” that showed no appreciable metabolite production by removing the BGC for biosynthesis of solanapyrones from the wild-type As. rabiei (FIG. 4B). Then, the present inventors introduced the atr1 cloned from the S. alpinum gDNA into the “clean host”. Expression and correct intron splicing of the atr1 were confirmed by RT-PCR analyses (see FIG. 7). The LC-MS/MS and NMR analyses indicated a known lichen depside, 4-O-demethylbarbatic acid (Compound 1) was produced by a strain expressing the atr1 (FIGS. 4B and 4C), suggesting that two 3MOA units were joined into a depside by the Atr1 itself.

As the biosynthesis of atranorin from compound 1 requires oxidation of a methyl group and methylation of the carboxyl group within each of the two 3MOA units (FIG. 4A), the present inventors individually introduced genes encoding the atr2 and the atr3 to the atr1-expressing strain. Co-expression of the atr1 with the atr3 yielded 7′-O-methylated analogue of compound 1, proatranorin I (Compound 2), indicating that the Atr3 is indeed a carboxyl methylase, as is the Trt5 in A. terreus (FIGS. 4B and 4C). The LC-MS/MS analysis showed that co-expression of the atr1 with the atr2 yielded compounds 3 and 4, which were annotated as a hydroxylated analogue of compound 1 (proatranorin II) and a further oxidized aldehyde (proatranorin III), respectively (FIGS. 4B and 4C). NMR analysis on the isolated proatranorin III (Compound 4) confirmed that the oxidation by the Atr2 occurs at C-9 of compound 1. Finally, the present inventors generated a strain expressing the three genes, the atr1, atr2 and atr3. The production of atranorin (Compound 5) was confirmed by MS/MS spectral matching with the reference spectrum of atranorin in the Lichen Database. It was also confirmed by comparison of the chemical profile with an authentic voucher specimen for the sequenced S. alpinum that produces atranorin (compound 5) and lobaric acid, as a cortical and medullary substance, respectively (FIGS. 4B and 4C). Intriguingly, MS/MS spectral matching of compound 6 identified it as another lichen depside, baeomycesic acid, which has not been reported from S. alpinum in nature, suggesting that the Atr3 exhibits relaxed substrate specificity in the heterologous host.

### Example 22: a Novel Phylogenetic Clade of PKS Responsible for Biosynthesis of Lichen Substances

To study the evolutionary relationships of fungal NR-PKS and the Atr1 responsible for atranorin production, the present inventors reconstructed a phylogenetic tree of the concatenated sequences of conserved KS and product template (PT) domains of 103 NR-PKSs found in the six Cladonia spp. and S. alpinum, and 82 NR-PKSs that have been linked to known compounds in non-lichenized fungi. Fungal NR-PKSs were hitherto largely classified into eight groups (Groups I-VIII) in previous phylogenetic analyses (7, 8, 42-44). Here, the present inventors identified a novel NR-PKS group (Group IX) supported by 100% bootstrap, which contained the PKS23 family as well as the PKS1 and PKS2 families (FIG. 5). The PKS1 family was previously proposed to biosynthesize 3MOA-derived lichen substances in C. rangiferina. For lichen NR-PKSs in the Group IX, all but the PKS2 in S. alpinum possessed a C-methyltransferase (cMT) domain involved in the methylation of polyketide intermediates. Intriguingly, the newly identified Group IX included three previously characterized NR-PKSs, AscC, StbA and TmPKS12, in non-lichenized fungi (FIG. 5). These NR-PKSs lack cMT domain and are known to produce orsellinic acids. Moreover, it is noteworthy that the Group VIII basal to the Group IX included several NR-PKSs that have been linked to orsellinic acid-derived compounds in mushroom-forming fungi (Agaricomycetes) and Fusarium graminearum (FIG. 5), indicating that the PKS1 and PKS2 families are likely involved in biosynthesis of lichen substances derived from a methylated form of orsellinic acid, as with the PKS23 family.

### ACCESSION NUMBER

Depository institution: Korea Agricultural and Biotechnology
Research Institute (KACC)
Accession number: KACC83048BP
Accession date: May 28, 2021

1. An atranorin biosynthesis gene consisting of one or more selected from the group consisting of SEQ ID NO: 1, SEQ ID NO: 3, and SEQ ID NO: 5.

2. The atranorin biosynthesis gene according to claim 1, wherein the atranorin biosynthesis gene is derived from a lichen.

3. The atranorin biosynthesis gene according to claim 1, wherein the lichen is Stereocaulon alpinum.

4. A recombinant expression vector for producing a lichen-derived metabolite, comprising an atranorin biosynthesis gene consisting of one or more selected from the group consisting of SEQ ID NO: 1, SEQ ID NO: 3, and SEQ ID NO: 5.

5. The recombinant expression vector for producing a lichen-derived metabolite according to claim 4, wherein the lichen-derived metabolite is one or more selected from the group consisting of 4-O-demethylbarbatic acid, proatranorin I, proatranorin II, proatranorin III, atranorin, and baeomycesic acid.

6. A recombinant microorganism transformed with the recombinant expression according to claim 4.

7. A method of producing a lichen-derived metabolite, comprising a step of culturing the recombinant microorganism according to claim 6.

8. The method of producing a lichen-derived metabolite according to claim 7, wherein the lichen-derived metabolite is one or more selected from the group consisting of 4-O-demethylbarbatic acid, proatranorin I, proatranorin II, proatranorin III, atranorin, and baeomycesic acid.

9. An Ascochyta rabiei ATR-11 strain (Accession Number: KACC 83048BP).

10. The strain according to claim 9, wherein the strain is for producing atranorin.

11. The strain according to claim 9, wherein the train is a strain into which an atranorin biosynthesis gene consisting of one or more selected from the group consisting of SEQ ID NO: 1, SEQ ID NO: 3, and SEQ ID NO: 5 is introduced.

12. A method of producing atranorin, comprising a step of culturing the strain according to claim 9.


SEQUENCE LISTING (46 sequences)

SEQ ID NO: 1 - DNA, 7808 bp, Artificial Sequence
  Stereocaulon alpinum atr1 gene(MZ277879)
atggcgtctaaccatgaggagctaccttcaatggtagtgttcagcccgcagagcaaagccccaaaagaaggatacctcgatgagttacggtcatatctatgcggcaaggctgaacttcgacctttgcttgatggtatcgaaaaccttccaaacacctggtcaatctttgctcaaaggaactccgacattgcagctctgactcaaggaatccggtacacacaggcactctcagactgggccaagcatggaacttcgtcaggaatctcgaacgttatgtctgggatcctatcgttgcctctcttgaccatcattcaggtggtccaatacttccaattccttgaagtgaaaaagctccgtcacagcgacttcatggagcgactgaggtgtaggggtggagttcaagggtattgtggcggcctaatgcctgccattgccatcgcctgctctgcgacagaagctgaggttgttaccaacgctgttaaagcgatgggtattgcactaggtgttggcgcttacggggagcttggggacgatgagaacgtcctagggccaactacaatagttgtccgcctcaagcaggagggccaaggtggagacatcataaaggactttcctgacgtgagctgctcgttccgcacctaccaaactcactaactttgttacaggcgcatatatcagctatcacggacccaaagaccgtcagcattgtagggtcagccccatctttggcagagatacaagcccgagtgaagtctaacggcatgcaaacccaagccatgcatctgcgtgggaaagtgcataatcccgaaaacgctaatctagcgctggagctttgcatgctttgcgacgagcatgaggaactctctttgccaaacgcttcacacttgacggctcctttgagatctaacaagacagggaagaagttattagatgtctctcttactcatgaagcgatcgaaacaatcctggcatcctgctgtcaatggtacgaccttctcaaaggggtgtgcaaagacttggagaaaacaggaactcaatcacaccttttcgcatcgttcggcattggcgactgcatccctctgacaccgtttcatcaggcagggctacagattaccaaattggacgttctctcctttgtcaaagccttgatgccgcctgttctaccaatgtccggtcacaaccaccaatatgcttatccaactgacgctgtggctgtcgtcggaatggcgtgtcgcctaccgggtgccaacagcgtcgaagaactctgggatctgatctcgtccggtggttcaactgttacccctgtcccagaagatcggatggatattgcaggtagctttcgagctatgcaagatccgaagtgggctgctaagcaacaatggtggggcaattttatctctgatattgccggatttgaccacagcttcttccgcatgagccctcgggaagccgcatcgatggatccacaacaacgcattctcctcgagacagcataccaagcaatggagtcgagtggctaccttggttcgcatcgacgcgagtcaggtgatccagtgggagtattcctaggagcaagtttcgtcgagtacctggacaatacttcctcaaacccaccgacagcatacacctccactggcacaatcagggcgtttttgagcggcaagataagttattactttggctggaccggtccttctgaaattctcgacacagcatgctcgtcgtctctggttgccattaacagggcctgcaaggcgattcagaacgatgagtgccccatggcacttgctggtggcgttaatctaattactggtatccacaattatttggatctcgcaaaagcaggcttccttagtcctagtggacaatgcaagccgtttgatggcgctgctgatggctactgccgttcggaaggcgcgggtctcgttgttctcaagcggttgagccaggcattgaccgatgggaatcagatccttggcgtcatcacgggggctagcaccaatcaaggcggtttaagtccatcgctcacagtcccgcacagcgctgcccaggtcaaattgtaccaaaacattcttcatcaggcgggaatgagaccggagcaagtttcttattgcgagactcatggcactggcactcaagcaggcgatcctctggagattgagagtgtgcgggaggtttttggtggtcctaaaagacaggattccatgcacataggctcaatcaaaggcaacattggtcactgtgagactgctgcaggagtcgctggccttctcaaagcgctcgtcatggtcaacaaagctgcaataccgcctctagcgagtcacaagagcttgaatccgaagattgccgccctcgaacctgataagcttgctatttcaagttgtcttgaagactggagagtctcgccgagagctgcattggtaaacagctatggcgcggccggaagtaattctgctgtcctactatgccaggcccctgacattgacaacgcacctttacaccgcgtagcaaccacagagcatacctatccaatcatactgagcgctgcatcgaaaccaagtctactttcgaatgctgaaaacatagcaagctatttgaggaaagcaacttccaagtgcacgattgccgatgttgcgttcactctcgtcaaacaacgtaaacgccatccgttacaatggataacaatggagtcaagcatcgacggcttggtcaagtccctggggtctttgcaggacccctcaaatgctccgcagccaaagaaggtggttatgacattttctggccaaagtcgacaatcgattgggctcaacaaagaatggtatgactctttccctctgttccggcgccatgtgaacgaatgcgacgacttgctgcagcagagcggatttccttcctgcaagtctgcaatgttcgataaggagcctgctcgggatgttgtgccactccagtgtgcgatgtttgctgttcagtacgcatctgccttgtcttggatcgattgtggacttcaagtagaagctgtaattggccacagcttcggtgagttgacagctttggcagtgtcgggtaccctttccctcaaagatgctctcaatcttgttgctacccgagctacattgatgcaatcaaaatgggggcctcacaaagggaccatgctcttgatcagtgcggctactgagatggtacgtaaaatcattgcaggaaaccgcgatgtcgagatcgcttgccacaatgcaccaacaagccaaattgttgtaggtacgcaagctgctatctcggaggttgaaaaagttctcgaaaacaatacggagtatcgcggcatccagagtcagcgcctcaatgttacgcacggcttccattctcaattcaccgagccattactcgagaatctttcagagagtgccagatctctagtcttccatgagccgaagattttgctagagtgttgcactcttgaagaactgaaccatgtggggcctgatcacttagcacgacacactcgggagcctgtttacttctaccacgctgtacgccgcctcgagcagcgtcttggcacatgtctgtggcttgaggctggtttcgactctccgattataccaatgaccaagagggccgttgaattcccagagagacacacctttctggacatgaagactccgagtgggacgaatcccactaagatgctcactaccgcaacgatcaacctttggcaaaacggtgctacatcctccttctggggcttccatcccatagagacaacaaacattaaacaggtgtggctgccaccgtaccaattcgatcggacctcccattggatgccttataccgatcatgctttggagatgagcaaaatccaagccgtcatcagcaattctgaacccttggtcgaattatcaactaagccaccgcgactggttgagcctcggacgaagcccagtgagaaaggtgaattctccatgaacacacaagcgcgacgctataccgaaattgtttctggtcatgctgttttgagtcgaccattgtgtcctgcagctatgtacatggaatgcgcaatcatggccgcacagttgtctattggcaacatcgtgggccaggcgccgtggttcgagaatctgacctttgaggctcccctcggaatggatcctgacaacgacaccacagtggtcttgaaggatgatggttccaaaagcagatggtcatttgtcgcgaggagcacgagtaggagcaacccgaaacgcaagccagtcttgcacgccaaaggggacttcggtttcactacacaaactcaagttcatcgatatgagaggcttgtaaccgaccgcatgaggcatttgcagcactctaaaagcgagacacttaagtccaagcgagcttatggacttttctcccgaatcgttcgttatgcagagctattgaagggaatttcatcaattactctaggtgattcagaagcatctgcaataatcgacgtaccgcttggcgccagcaccgaggacagctcggcaacaggactctgtgattgcgtggcacttgatgccttcattcaagtggtcggcttattgatcaactccggtgatgattgtgccgaagatgaggtttttgttgcaactggtgtcgagaacttctcaatgtcgttggcctgcgacttcgatcgctgcagaacttggcttgtattcgccatgttcacaccatcgggtaatggtaaagcgatgggagatgttttcatcctgacgagggacaatgtcctggtcatgacaatcatgggtgtacaatttaccaagcttccgattacaaggctggagaagctattggactcggccaatcccaaagctcacaatacgccaattttgaagtcaagtcagcaagactccatcgtatcggccagcagtagctctagtacggagcatagcgatgacgacagcgaggacgatggatcacggtctcccagccacagtgatacctccgttgacagcgagtcagaagcgccagcggacaacggtgctgcaaagaaactaaagtctctcattgcatcatatgttgggatcgctgaagacgctatttccgatgatgcaaatattgcggatcttggagtcgactctcttgctgctacagaactagccgacgagatctcgaacgacttcgctaaggaaatcgacggcggcgagcttcccatgatgacatttggtgaactttgtcgaatagtggccccggagatggctgcaaagcctgcaaaggcgaaaaagaagattccctacaaaggtaaagatgaagcgactgttgttgagtcacacccaggtaaatcacagagtgagatcaaggacttgaaggctgtggtggagcctttgccaagatcgactcccatgttatcagataccaccgtcgtccgatctgatcctacccaagtccttcgccaaattgataccatgtttcagtcatctgctgatacattcggcttcacggactactggacagcggtcgccccaaagcagaataagttagtcctggcatatattggcgaggagttcagaaagctaggactcgatctctgggcggttcaacctggagcaacccttccacatattgagtaccttccaaagcatgaaaaggttgtccagagactgtgggatattcttgctgatcatggtattgtctacaattatgacgccgcaaaggtgcggtccagcaaacctcttccggacgctccttccacggctctcctcaatgagctcaacgccttgttcccaaatttcgccaatgagaatcggctaatgtctgtcacggcgcctcatttcgcggatggtttgaggggaaagactgatcatattagtctgctgtttgggagtcaacgtggccaggaatgcctgaatgacttctataacaattcgcctcagttggccgtgatgaccgatcacttgctcactttctttaagcagctcctgaaagaagccccacttgaaggttcactccgaatccttgaggtcggtggcggctttggagggacgacaaaacgactagcagaaatgctggaggcactaggtcagccggtcgagtatacgttcacggatgtctcgtccatgctagtgaaggaagcaaggaaaaagttttccaaacattcttggatggactttcagtccttgaatcttgaaaaggaccctccagcttctcttcaaaggacatacgatattgtgattgggactaatgtagtgcacgcaacatcgaacattgtgaattcgacgacacgcatgcgaagtcttttgaggaagggtggctttatcgtcttatctgaagtcacccgcattgttgactggtacgatctggtttatggactccttgatggctggtgggccttcaaagactctaggacgtatcctttgcagcctgccgatgactgggtgagagacctcatgaaagcgggattcgagaccgcatcctactcgaggggagactccgaggagtccaacacacagcagctgattattggctcgaccaggccaagcaaggtagcgtctaccagcgggctttcggaagccaggctaagtaagtcctatagaattgagacaatgccatacaagatcatcgacgacacggagatactggcggacgtcttcttcccagagcatgaagtggcctcggaagccatgcctataggtaatcttctccagtgtgtctagcaatctcacgcagccgctgacttctgacaagccctcatgatccacggtggcggttttatgacactctccaaaacggccatcagaccctaccagacccaattcctcgtggaaaacggctatcttcctatcagcatcgattaccgactttgccccgagattgatctaatcgctggtccgatgacggacgttcgtgacgctctaacctgggtgcgaaaacagctgcctgcgattgctcgtacccgcggcatcaatgtggacccaacgaaagttgtagtcatcggatggtccacaggtggtcatctggccctgactacagcgtggacatgtgaggacattggagaagagccacctgttgcagtccttagcttctacggtccaacgaacttcgaatctgaaggtgcgcgtccattgacactcttttctgggtgctagtgccacctgtggtccaaaccgtagcttaggctgcattcctacaagatcgctaattgttcataacagacattgatcggaggcgtgcggaacagtacccagaacggactatgtcgttcgatcggatccgcaagtcattatccacgaagcctgtgagtatctccttgctattgcaaattctcctaggccttgacattgcttccccatagcattgttactcttctactcactgctaataccctcttccacacgcagatcacttcctatgactgtcccaccggcacagacagcaccggtctcggctgggtgcgtcccggcgacccacgctctgaactcgtcttgtccctcttcaaagaatctcacggtctgcaagtgatgcttaacggtctttctgccgcagatttggccaaacctccgccattggctaaaattcaggcgatcagcccgatggcgcagctcaaggccgggaggtacaacgtcccaacttttgtaattcattcagattgtgatgagatcgcgccgttcagggatagcgaggcttttgtggaggaactggcgaggagaggggtgaagactggactagggagggtgaggggcaagaagcatattcatgacttggcgttgaagccggagaaggacggatgggtggacggtgcgggcgttggatatgagttcattttcgatgttgttggacgaggcgttaggggttga

SEQ ID NO: 2 - protein, 2500 aa, Artificial Sequence
  Stereocaulon alpinum atr1(QXF68953)
MASNHEELPSMVVFSPQSKAPKEGYLDELRSYLCGKAELRPLLDGIENLPNTWSIFAQRNSDIAALTQGIRYTQALSDWAKHGTSSGISNVMSGILSLPLLTIIQVVQYFQFLEVKKLRHSDFMERLRCRGGVQGYCGGLMPAIAIACSATEAEVVTNAVKAMGIALGVGAYGELGDDENVLGPTTIVVRLKQEGQGGDIIKDFPDAHISAITDPKTVSIVGSAPSLAEIQARVKSNGMQTQAMHLRGKVHNPENANLALELCMLCDEHEELSLPNASHLTAPLRSNKTGKKLLDVSLTHEAIETILASCCQWYDLLKGVCKDLEKTGTQSHLFASFGIGDCIPLTPFHQAGLQITKLDVLSFVKALMPPVLPMSGHNHQYAYPTDAVAVVGMACRLPGANSVEELWDLISSGGSTVTPVPEDRMDIAGSFRAMQDPKWAAKQQWWGNFISDIAGFDHSFFRMSPREAASMDPQQRILLETAYQAMESSGYLGSHRRESGDPVGVFLGASFVEYLDNTSSNPPTAYTSTGTIRAFLSGKISYYFGWTGPSEILDTACSSSLVAINRACKAIQNDECPMALAGGVNLITGIHNYLDLAKAGFLSPSGQCKPFDGAADGYCRSEGAGLVVLKRLSQALTDGNQILGVITGASTNQGGLSPSLTVPHSAAQVKLYQNILHQAGMRPEQVSYCETHGTGTQAGDPLEIESVREVFGGPKRQDSMHIGSIKGNIGHCETAAGVAGLLKALVMVNKAAIPPLASHKSLNPKIAALEPDKLAISSCLEDWRVSPRAALVNSYGAAGSNSAVLLCQAPDIDNAPLHRVATTEHTYPIILSAASKPSLLSNAENIASYLRKATSKCTIADVAFTLVKQRKRHPLQWITMESSIDGLVKSLGSLQDPSNAPQPKKVVMTFSGQSRQSIGLNKEWYDSFPLFRRHVNECDDLLQQSGFPSCKSAMFDKEPARDVVPLQCAMFAVQYASALSWIDCGLQVEAVIGHSFGELTALAVSGTLSLKDALNLVATRATLMQSKWGPHKGTMLLISAATEMVRKIIAGNRDVEIACHNAPTSQIVVGTQAAISEVEKVLENNTEYRGIQSQRLNVTHGFHSQFTEPLLENLSESARSLVFHEPKILLECCTLEELNHVGPDHLARHTREPVYFYHAVRRLEQRLGTCLWLEAGFDSPIIPMTKRAVEFPERHTFLDMKTPSGTNPTKMLTTATINLWQNGATSSFWGFHPIETTNIKQVWLPPYQFDRTSHWMPYTDHALEMSKIQAVISNSEPLVELSTKPPRLVEPRTKPSEKGEFSMNTQARRYTEIVSGHAVLSRPLCPAAMYMECAIMAAQLSIGNIVGQAPWFENLTFEAPLGMDPDNDTTVVLKDDGSKSRWSFVARSTSRSNPKRKPVLHAKGDFGFTTQTQVHRYERLVTDRMRHLQHSKSETLKSKRAYGLFSRIVRYAELLKGISSITLGDSEASAIIDVPLGASTEDSSATGLCDCVALDAFIQVVGLLINSGDDCAEDEVFVATGVENFSMSLACDFDRCRTWLVFAMFTPSGNGKAMGDVFILTRDNVLVMTIMGVQFTKLPITRLEKLLDSANPKAHNTPILKSSQQDSIVSASSSSSTEHSDDDSEDDGSRSPSHSDTSVDSESEAPADNGAAKKLKSLIASYVGIAEDAISDDANIADLGVDSLAATELADEISNDFAKEIDGGELPMMTFGELCRIVAPEMAAKPAKAKKKIPYKGKDEATVVESHPGKSQSEIKDLKAVVEPLPRSTPMLSDTTVVRSDPTQVLRQIDTMFQSSADTFGFTDYWTAVAPKQNKLVLAYIGEEFRKLGLDLWAVQPGATLPHIEYLPKHEKVVQRLWDILADHGIVYNYDAAKVRSSKPLPDAPSTALLNELNALFPNFANENRLMSVTAPHFADGLRGKTDHISLLFGSQRGQECLNDFYNNSPQLAVMTDHLLTFFKQLLKEAPLEGSLRILEVGGGFGGTTKRLAEMLEALGQPVEYTFTDVSSMLVKEARKKFSKHSWMDFQSLNLEKDPPASLQRTYDIVIGTNVVHATSNIVNSTTRMRSLLRKGGFIVLSEVTRIVDWYDLVYGLLDGWWAFKDSRTYPLQPADDWVRDLMKAGFETASYSRGDSEESNTQQLIIGSTRPSKVASTSGLSEARLSKSYRIETMPYKIIDDTEILADVFFPEHEVASEAMPIALMIHGGGFMTLSKTAIRPYQTQFLVENGYLPISIDYRLCPEIDLIAGPMTDVRDALTWVRKQLPAIARTRGINVDPTKVVVIGWSTGGHLALTTAWTCEDIGEEPPVAVLSFYGPTNFESEDIDRRRAEQYPERTMSFDRIRKSLSTKPITSYDCPTGTDSTGLGWVRPGDPRSELVLSLFKESHGLQVMLNGLSAADLAKPPPLAKIQAISPMAQLKAGRYNVPTFVIHSDCDEIAPFRDSEAFVEELARRGVKTGLGRVRGKKHIHDLALKPEKDGWVDGAGVGYEFIFDVVGRGVRG

SEQ ID NO: 3 - DNA, 1761 bp, Artificial Sequence
  Stereocaulon alpinum atr2 gene(MZ277878)
atggctctcctagacacaattgaactgttctccaacttctcccttagtggagtgttcgctggcctagttctagcttctctcctcgtaagttcaaccactgttacgtcgtcaaaatgtgccagccttccctttgcgagaactgacacataagcaactagaccacaacttactgcatttggaacatcttctacaatatttaccttcatcccttgaaaggttaccctggcccaaaattcctcacaacttctcggcttccatatctcaaatggatgttctcaggaacccttgtgcccaatttccaaaggctccatgagcagtacggtcctgttgttcgtgttgcgcccaacgagctctcgtacatcaacccggaagccttgaaaaccatctacggccaccgtcaaccaggcgaggggtttcgcaagaatccagcattcttccagccagctacgaatggcgttcattccatcttgacctccgaaggtgacgctcatagtagtgtccgccggaagatcctgcccgctttctcggataaggcgcttgcggaacagcaggacatcctccagcatttcacagatctgttgatccggaaattgcgcgagagagtcgaagcatccaaaagctcagaacctgttgacatgttcgaatggtacatctggactaccttcgacttgattggagatctcgcatttggcgagccgttcaactgcctcgaagcagctagctttacagaatgggtcgcccttgtgttcaacgcgtttaagacttttgcgttcatcaacatcaggtaagcaaggtagactcaccccgcaacgaataaaagaaacgcgcttacgaaaccgaactagcaaacagctagctccccttgacaagctagtcagactgatgatcccaaagtccatgaaagcccggcaagacaaagtcttctccctgaacgtcgccaaagtcgaccgccgcattgcttccaaagcagaccgtccagacttcctatcgtacattattaagggcaaggacggggtagcaatggcacttccagaactctacgcaaattccacgctccttgtcctcgccggtagcgagagcacagcatctggcttggctggcattacctttgagctactcaagcaccgagaggctcagaagaaggccgtggaagagatacgcagcgctttcaaaaccgaagatgagattgtacctgagagcgtcaagcgacttccataccttgcggccatggtttccgaagggctccgaatgtatccgcccttcccagagggacttcctcgtcttacaccacggcaaggggctcagatctgtggccaatgggtccctggaggagtaagctatgacccccatcctccgggactagctaatcttctcatatagacatatgtgcaatttagcacccacgccgctcaccgcgccagcgccaattttacagacccgaatgtcttcgcccctgagcgctggcttggcgacacgaaattcgcgtcggatatcaaagaagcttcgcagcccttctcgataggtccacgtagctgcattggcaggaagtgagtactttgttcgcctcatttgcacgaagagtactaattatacgggaaacccagtcttgcgtaccttgaaatgcgcttgattctagcgcgtatgttgtggagcttcgacatgcaactcactcccgaatgcgaagactgggacgaccagaatagctggatccagtgggacaagaaacctttgatggtgaagttgtcgcttgtcaaaagatag

SEQ ID NO: 4 - protein, 506 aa, Artificial Sequence
  Stereocaulon alpinum atr2(QXF68952)
MALLDTIELFSNFSLSGVFAGLVLASLLTTTYCIWNIFYNIYLHPLKGYPGPKFLTTSRLPYLKWMFSGTLVPNFQRLHEQYGPVVRVAPNELSYINPEALKTIYGHRQPGEGFRKNPAFFQPATNGVHSILTSEGDAHSSVRRKILPAFSDKALAEQQDILQHFTDLLIRKLRERVEASKSSEPVDMFEWYIWTTFDLIGDLAFGEPFNCLEAASFTEWVALVFNAFKTFAFINISKQLAPLDKLVRLMIPKSMKARQDKVFSLNVAKVDRRIASKADRPDFLSYIIKGKDGVAMALPELYANSTLLVLAGSESTASGLAGITFELLKHREAQKKAVEEIRSAFKTEDEIVPESVKRLPYLAAMVSEGLRMYPPFPEGLPRLTPRQGAQICGQWVPGGTYVQFSTHAAHRASANFTDPNVFAPERWLGDTKFASDIKEASQPFSIGPRSCIGRNLAYLEMRLILARMLWSFDMQLTPECEDWDDQNSWIQWDKKPLMVKLSLVKR

SEQ ID NO: 5 - DNA, 1254 bp, Artificial Sequence
  Stereocaulon alpinum atr3 gene(MZ277877)
atgacttccgttgatacaatgcctccaccgatggttcgcctcgagagtcagcctgacgacttgatgggctcttctgatgtcgcggacgtttcagaccttcttccaggccatacaaatggactcgaggatgaagtgaaaattccagcaaccaatggactcaagagccatccagtggttacgacaggaacagaaaaaaccggtgtgatgccgccccttcagcccgaaagcaagaagaacaacaaaggcgttccgtggtaagacctggacatacaccgctctttttgtcaagccatgactgttacgacttcctgttgttttctatcatttctctgccagtagtgctgaggtagacaggtatcacgcctcaccgaacgatatcgatcccgtgactcgcggtctgctcgaaaattacagcaaaatcccctcggatcaagtacagcagcacgttatcgcaattgtaagtttccaaaggttactccttgatgttctgcaaccggattgcagttggcgcactagatggctctctccctagacaagcatcgctaaaaccgtctttttctcccaaaacagcgcgaaaaagcctgggacgtctacccctacccctgcatcggccaattcctcttcctgaacctaaccataaacctctccccctactacccgtccctcgtctcccgcctccgcgatcaaaatcaaacactcctcgacctcggctgctgcttcgcccaagacgtccgcaaactcgtctctgatggcgccccgagtcaaaacatctacggcgccgacctctatggcgagttcatggatctaggctttgaactctttcgcgaccgaaagacgctcaaaagcacgtttttcccgactgatatactaaacgagcgggatctgctgttgaagggcttggatggcgagatggatgttgtttatttggggctgtttttgcatcattttgattttgagacttgtgtcaaagtttgtacgcgcgttactaggctcctgaagcccaagccggggagtctggttatgggggtccaggtggggagtttggtgggtgatactaaacccattcctattcctagtggtgggattttgtggaggcatgatattgcgagtcttgaaagggtttgggaggaggttggagcgctgacggggacgaagtggaaggttgaggctaggttggagaggggcaaagggttcggggagaagtggcagcttgaggggacgaggaggttggggtttgaggtttataggctttga

SEQ ID NO: 6 - protein, 346 aa, Artificial Sequence
  Stereocaulon alpinum atr3(QXF68951)
MTSVDTMPPPMVRLESQPDDLMGSSDVADVSDLLPGHTNGLEDEVKIPATNGLKSHPVVTTGTEKTGVMPPLQPESKKNNKGVPWYHASPNDIDPVTRGLLENYSKIPSDQVQQHVIAIREKAWDVYPYPCIGQFLFLNLTINLSPYYPSLVSRLRDQNQTLLDLGCCFAQDVRKLVSDGAPSQNIYGADLYGEFMDLGFELFRDRKTLKSTFFPTDILNERDLLLKGLDGEMDVVYLGLFLHHFDFETCVKVCTRVTRLLKPKPGSLVMGVQVGSLVGDTKPIPIPSGGILWRHDIASLERVWEEVGALTGTKWKVEARLERGKGFGEKWQLEGTRRLGFEVYRL

SEQ ID NO: 7 - DNA, 27 bp, Artificial Sequence
  Sol1PpDS33_fwd
agatctcggcacgttttgatcgcaaag

SEQ ID NO: 8 - DNA, 32 bp, Artificial Sequence
  Sol1PpDS33_rev
aagcttagtaatatctggcgatgaagcgtcag

SEQ ID NO: 9 - DNA, 38 bp, Artificial Sequence
  Inf_Sta046440_fwd
cagatattactaagctcatggcgtctaaccatgaggag

SEQ ID NO: 10 - DNA, 44 bp, Artificial Sequence
  Inf_Sta046440_rev
gtaacgttaagtggatccttacaactaaagctctcaatcaaccc

SEQ ID NO: 11 - DNA, 20 bp, Artificial Sequence
  Sta046440_check1F
ggacgatgagaacgtcctag

SEQ ID NO: 12 - DNA, 19 bp, Artificial Sequence
  Sta046440_check2F
cgagtcaggtgatccagtg

SEQ ID NO: 13 - DNA, 19 bp, Artificial Sequence
  Sta046440_check3F
ccaatcatactgagcgctg

SEQ ID NO: 14 - DNA, 19 bp, Artificial Sequence
  Sta046440_check4F
cacatgtctgtggcttgag

SEQ ID NO: 15 - DNA, 18 bp, Artificial Sequence
  Sta046440_check5F
caactccggtgatgattg

SEQ ID NO: 16 - DNA, 20 bp, Artificial Sequence
  Sta046440_check6F
caacccttccacatattgag

SEQ ID NO: 17 - DNA, 23 bp, Artificial Sequence
  pII99_sol5_fwd
gaattcagagcgacagtgaaagc

SEQ ID NO: 18 - DNA, 37 bp, Artificial Sequence
  pII99_sol5_rev
agatctagctagcttggcgtgatgtcagtcaatgctg

SEQ ID NO: 19 - DNA, 27 bp, Artificial Sequence
  pII99_tef1a_fwd
gaattccagccgagacagcagaatcac

SEQ ID NO: 20 - DNA, 41 bp, Artificial Sequence
  pII99_tef1a_rev
agatctagctagcgtgttatgttttgtggaatataaaaggg

SEQ ID NO: 21 - DNA, 40 bp, Artificial Sequence
  Inf95_Sta04642_fwd
atcacgccaagctagcacgatgacttccgttgatacaatg

SEQ ID NO: 22 - DNA, 41 bp, Artificial Sequence
  Inf98_Sta04642_fwd
aaacataacacgctagcacgatgacttccgttgatacaatg

SEQ ID NO: 23 - DNA, 39 bp, Artificial Sequence
  Inf99_Sta04642_rev
ggagaccggcagatcccgtcacatgtccgtcataataac

SEQ ID NO: 24 - DNA, 42 bp, Artificial Sequence
  Inf98_Sta04643_fwd
aaacataacacgctagaaccatggctctcctagacacaattg

SEQ ID NO: 25 - DNA, 41 bp, Artificial Sequence
  Inf95_Sta04643_fwd
atcacgccaagctagaaccatggctctcctagacacaattg

SEQ ID NO: 26 - DNA, 39 bp, Artificial Sequence
  Inf99_Sta04643_rev
ggagaccggcagatcgaggagcaaaggaacaaggaacag

SEQ ID NO: 27 - DNA, 22 bp, Artificial Sequence
  pII99_MK5
cgtcaagagacctacgagactg

SEQ ID NO: 28 - DNA, 25 bp, Artificial Sequence
  pII99_MK3
gctatacttctaggtcttggaagag

SEQ ID NO: 29 - DNA, 38 bp, Artificial Sequence
  Inf_BLE_pII99_fwd
gtaggtctcttgacgcattaagacctcagcgctagtgg

SEQ ID NO: 30 - DNA, 39 bp, Artificial Sequence
  Inf_BLE_pII99_rev
gacctagaagtatagcggtgttacggagcattcactagg

SEQ ID NO: 31 - DNA, 23 bp, Artificial Sequence
  L5
gaggtcttcacaccacaagttcg

SEQ ID NO: 32 - DNA, 52 bp, Artificial Sequence
  L3
ggcaaaggaatagagtagatgccgaccgggctcttgcgtagtggtacagatg

SEQ ID NO: 33 - DNA, 51 bp, Artificial Sequence
  R5
gttgacctccactagctccagccaagcgaataggtaacaaagccagccgag

SEQ ID NO: 34 - DNA, 23 bp, Artificial Sequence
  R3
gagaattgcggcgcaggatgttc

SEQ ID NO: 35 - DNA, 24 bp, Artificial Sequence
  N5
ctgactttcttcttgcagccctgc

SEQ ID NO: 36 - DNA, 24 bp, Artificial Sequence
  N3
ctaacgatgtcgttagtcgccttg

SEQ ID NO: 37 - DNA, 21 bp, Artificial Sequence
  HYG-F
gcttggctggagctagtggag

SEQ ID NO: 38 - DNA, 24 bp, Artificial Sequence
  HYG-R
cggtcggcatctactctattcctt

SEQ ID NO: 39 - DNA, 25 bp, Artificial Sequence
  YG-F
cgatgtaggagggcgtggatatgtc

SEQ ID NO: 40 - DNA, 26 bp, Artificial Sequence
  HY-R
tgtagtgtattgaccgattccttgcg

SEQ ID NO: 41 - DNA, 20 bp, Artificial Sequence
  P1_fwd
ggacgatgagaacgtcctag

SEQ ID NO: 42 - DNA, 20 bp, Artificial Sequence
  P1_rev
gagagttcctcatgctcgtc

SEQ ID NO: 43 - DNA, 22 bp, Artificial Sequence
  P2_fwd
catcgacgacacggagatactg

SEQ ID NO: 44 - DNA, 23 bp, Artificial Sequence
  P2_rev
gaccgtgagattctttgaagagg

SEQ ID NO: 45 - DNA, 22 bp, Artificial Sequence
  Actin1_fwd
caatggttcgggtatgtgcaag

SEQ ID NO: 46 - DNA, 22 bp, Artificial Sequence
  Actin1_rev
gaagagcgaaaccctcgtagat